(19) United States
(12) Patent Application Publication
(10) Pub. No.: US 2009/0017512 A1
(43) Pub. Date: Jan. 15, 2009

(54) APPARATUS AND METHODS FOR THE PRODUCTION OF ETHANOL, HYDROGEN AND ELECTRICITY

(76) Inventors: Harold D. May, Mount Pleasant, SC (US); Tsutomu Shimotori, Mount Pleasant, SC (US)

Correspondence Address: FULBRIGHT & JAWORSKI L.L.P., 600 CONGRESS AVE., SUITE 2400, AUSTIN, TX, 78701, US

(21) Appl. No.: 11/951,745

(22) Filed: Dec. 6, 2007

Related U.S. Application Data
(60) Provisional application No. 60/868,933, filed on Dec. 6, 2006.

(51) Int. Cl. C12P 7/06; C12N 1/20

(52) U.S. Cl. 435/165; 435/252.3; 435/161

(57) ABSTRACT

The compositions, methods and apparatus of the present invention allow the production of electricity, ethanol and hydrogen, and combinations thereof. In some embodiments, the invention provides a process for generating electricity or hydrogen comprising supplying a microbial catalyst and a fuel source to a microbial fuel cell or a bio-electrochemically assisted microbial reactor (BEAMR), respectively, under thermophilic conditions. In other embodiments, the invention provides a process of generating ethanol and electricity or ethanol and hydrogen comprising supplying a microbial catalyst and a fuel source to a fermentation vessel in operable relation with a microbial fuel cell or a BEAMR system, respectively, wherein the microbial catalyst has a cellulolytic activity, an ethanologenic activity, and an electricigenic activity. Other embodiments include compositions and apparati for practicing the invention.

This application claims benefit of priority to U.S. Provisional Application Ser. No. 60/868,933, filed Dec. 6, 2006.

This invention was made with government support under DE-FG02-07ER86319 awarded by the Department of Energy. The government has certain rights in the invention.

### BACKGROUND OF THE INVENTION

A. Field of the Invention

The present invention relates generally to the fields of microbiology, biochemistry, biotechnology and biofuels. In specific embodiments, the invention concerns compositions, methods and apparatus for the production from biomass of electricity, ethanol and hydrogen, and combinations thereof.

B. Description of Related Art

Electricity may be generated microbially in a fuel cell through the action of microorganisms, including those that donate electrons to an electrode (Logan et al., 2006). The electricity generating bacteria are referred to as electricigens, electrode-reducing bacteria, and anodophiles (Lovley, 2006; Rabaey et al., 2007). Microbial fuel cells (MFCs) may be applied toward the enhancement of wastewater treatment (Logan, 2005), the generation of electricity in remote locations, sensor operation, and battery charging. The microbes catalyze both the oxidation of an organic substrate, which may be waste material, and direct the resulting electrons to the anode of a MFC. Here a group of thermophilic microbial catalysts are described in a MFC process that may be adapted to produce ethanol, hydrogen, electricity, or a combination thereof. These products could then be used as stationary or transportation fuels (liquid fuels for internal combustion engines, hydrogen for hydrogen fuel cells, and electricity for direct electrical application or battery charging).

Biologically produced ethanol is increasingly being considered as an alternative for petroleum-based liquid fuels. Cornstarch is presently the primary raw material for commercial ethanol fermentation in the United States, but the yield of ethanol is limited by the amount of grain that can be produced, and the energy gained is modest when compared with the amount used to produce the corn and the ethanol (Hammerschlag, 2006). Lignocellulose, a plentiful and inexpensive renewable resource, is an attractive alternative feedstock for ethanol fermentation for the production of bioethanol. However, cellulosic fermentation to ethanol is inhibited by the end-products: ethanol, hydrogen, lactate and acetate (Lynd et al., 2005). When cellulosic fermentation to ethanol is done thermophilically, the volatile ethanol is driven off and is distilled. This purifies the product and removes it from the fermentation vessel so that it cannot inhibit further cellulose consumption. However, the remaining organic acids lower the pH and represent a loss of energy as non-fuel products and inhibit the overall fermentation of the cellulose. Expensive caustic may be added to neutralize the pH, but this does not eliminate the feedback inhibition by the acids and eventually leads to the build-up of inhibitory levels of cations added with the base. The process described here will produce ethanol and electricity from cellulose, in part by eliminating the inhibitory waste products.

Biohydrogen is hydrogen that may be generated by biological processes or from biomass. The biological processes include anaerobic fermentation (Logan et al., 2002) with bacteria such as Clostridium butyricum or Thermotoga neapolitana (Eriksen et al., 2008), photosynthesis with algae, cyanobacteria and bacteria such as Rhodobacter sphaeroides and Enterobacter cloacae (Melis and Happe, 2001; Prince and Kheshgi, 2005). Alternatively, methane biogas, formed by anaerobic microbial fermentation of organic matter, can be steam reformed into hydrogen gas. By applying the thermophilic biocatalysts of the process described herein to a modified MFC, hydrogen can be produced from cellulose and other biomass. Biohydrogen industrial plants are proposed with the idea that the hydrogen produced could be used in a PEM hydrogen fuel cell to power an automobile or a stationary power source.

### SUMMARY OF THE INVENTION

The compositions, methods and apparatus of the present invention allow the production of electricity, ethanol and hydrogen, and combinations thereof. In one embodiment, the invention comprises an isolated organic acid-consuming thermophilic electricigenic bacterium. An “isolated” bacterium is one which has been identified and separated and/or recovered from a component of its natural environment. Organic acids include, but are not limited to, acetic, octanoic, benzoic, parahydroxybenzoic, sorbic, ascorbic, citric, lactic, malic, fumaric, tartaric, propionic, succinic acid, ester acids and their salts, or mixtures thereof. In a particular embodiment, the bacterium is Thermincola ferriacetica strain Z-0001 (Zavarzina et al., 2007).

In another embodiment, the invention provides a process for generating electricity comprising supplying a microbial catalyst and a fuel source to a microbial fuel cell under thermophilic conditions wherein the microbial catalyst consumes the fuel source and generates electricity. The microbial catalyst may comprise one or more bacteria, for example Deferribacteres or Thermincola. In some aspects of the invention, the microbial catalyst may be resistant to autoclaving. Autoclaving is defined as the process of sterilizing an object by exposure to 121° C. or greater at a pressure of 15 psi or greater for at least 15 minutes. In some embodiments of the invention, the microbial catalyst may be resistant to autoclaving for 30 minutes. In a particular embodiment, the microbial catalyst comprises Thermincola.

The microbial catalyst may be any microorganism that will consume the fuel source and generate electricity under thermophilic conditions. The fuel source may be any biomass or organic waste that may be consumed to generate ethanol, hydrogen or electricity. Examples of a fuel sources for use with the current invention include acetate, sugars, cellulose, hemicellulose or chitin. Potential sources of cellulose may include corn stover, peach waste, plant residues, forest litter, chitin and switchgrass. Examples of suitable plant residues include stems, leaves, hulls, husks, cobs and the like, as well as wood, wood chips, wood pulp and sawdust. In some embodiments of the current invention, acetate is produced as a byproduct, which may be consumed by the microbial catalyst to generate electricity.

In particular embodiments, the process may be performed under thermophilic conditions. Thermophilic conditions exist between about 50-75° C. For the thermophilic processes described within, about 50-70° C., about 50-65° C., about 55-70° C., about 55-65° C., about 50-65° C., or about 55-60° C. are of particular utility.

A further embodiment of the present invention comprises a process of generating ethanol and electricity comprising supplying a microbial catalyst and a fuel source to a fermentation vessel in operable relation with a microbial fuel cell, wherein the microbial catalyst has a cellulolytic activity, an ethanologenic activity, and an electricigenic activity, wherein the fuel source is consumed and ethanol and electricity are generated. The fermentation vessel of the invention may be maintained under thermophilic conditions. In some embodiments, the microbial catalyst may comprise Clostridium thermocellum, Thermoanaerobacterium thermosaccharolyticum or Thermincola spp., or any combination thereof. In a further embodiment, the invention provides a process of generating ethanol and electricity comprising supplying a microbial catalyst having ethanologenic activity and a fuel source to a first fermentation vessel, wherein a spent fuel source is generated, supplying the spent fuel source and a second microbial catalyst having a cellulolytic activity and an electricigenic activity to a second fermentation vessel, wherein the second fermentation vessel is in operable relation with a microbial fuel cell and wherein the spent fuel source is consumed and ethanol and electricity are generated. This may be achieved, for example, by the process of simultaneous saccharification and co-fermentation. In this embodiment, one or both fermentation vessels may be maintained under thermophilic conditions. In a particular embodiment, the first microbial catalyst having an ethanologenic activity may be Zymomonas mobilis. In such an embodiment, the first fermentation vessel comprising the first microbial catalyst, Zymomonas mobilis, is maintained under mesophilic conditions, and the second fermentation vessel comprising the second microbial catalyst may be maintained under thermophilic conditions.

In one embodiment, the current invention provides a process of generating hydrogen comprising supplying a microbial catalyst and a fuel source to a bio-electrochemically assisted microbial reactor (BEAMR) system under thermophilic conditions, wherein the microbial catalyst consumes the fuel source and generates hydrogen. The microbial catalyst may comprise one or more bacteria, including Deferribacteres or Thermincola. In some aspects of the invention, the microbial catalyst is resistant to autoclaving, for example, Thermincola.

A microbial fuel cell may be modified to produce hydrogen. An example of such a microbial fuel cell is a BEAMR system. Such a system is described in U.S. Patent Publn. 2006-0011491, which is specifically incorporated herein by reference in its entirety. Broadly described, the system comprises a fuel source that is oxidized by bacteria which generate electrons and protons. A power source is connected to the microbial fuel cell and an additional voltage is applied. In one embodiment, this power source may be a second MFC. The electrons generated by the bacteria are transferred to the anode, and, through a conductive connector, to the cathode. Oxygen is substantially excluded from the cathode area such that protons and electrons combine at the cathode, producing hydrogen. In one embodiment, the system further comprises a microbial catalyst.

In a further embodiment, the invention comprises a process of generating ethanol and hydrogen comprising supplying a microbial catalyst and a fuel source to a BEAMR system, wherein the microbial catalyst has a cellulolytic activity, an ethanologenic activity, and an electricigenic activity; wherein the fuel source is consumed and ethanol and hydrogen are generated. In one embodiment, the system is maintained under thermophilic conditions. In particular embodiments, the microbial catalyst comprises Clostridium thermocellum, Thermoanaerobacterium thermosaccharolyticum, Thermincola, Deferribacteres or any combination thereof.

A further embodiment of the present invention provides an apparatus for generating ethanol and electricity comprising a fermentation vessel in operable relation with MFC. The MFC may comprise an anode and at least two cathodes, or alternatively may comprise at least two anodes and a cathode. The apparatus may further comprise an inflow line and a return line, wherein the inflow line and the return line communicate between the fermentation vessel and the microbial fuel cell. In some embodiments, a MFC recycle may be attached to a stirred tank reactor. In one embodiment, the system further comprises a microbial catalyst.

Yet another embodiment of the present invention provides an apparatus for generating ethanol and hydrogen comprising a chamber, a fuel source, a microbial catalyst, and a power source in connective relation with an anode and a cathode, wherein the anode and the cathode are located within the chamber. In one embodiment, the system further comprises a microbial catalyst.

The power source may be any thing that provides power to the apparatus. Power sources used for enhancing an electrical potential between the anode and cathode are not limited and illustratively include grid current, solar power sources, wind power sources. Further examples of a power source include a DC power source and an electrochemical cell such as a battery or capacitor. In one embodiment, the power source is a microbial fuel cell.

The anode may be comprised of any material that allows oxidation, such as graphite. See Rosenbaum et al. (2007); Qiao et al. (2007). The cathode may be comprised of any material that allows reduction. In some embodiments, the cathode further comprises a catalyst. The cathode catalyst may be comprised of any material that increases the rate of the reaction. For example, the cathode catalyst may be comprised of platinum or lead dioxide. See Yu et al. (2007) and Morris et al. (2007). Non-platinum cathode catalysts may also be used (Yu et al., 2007). For example, iron(II) phthalocyanine and cobalt tetramethoxyphenylporphyrin have recently been shown to serve nearly as well (Cheng et al., 2006; Zhao et al., 2005) and are far less expensive.

In particular embodiments, the system further comprises a membrane. The membrane may be comprised of an ion exchange membrane (IEM). Any suitable ion conducting material may be included in an IEM. For example, a perfluorinated sulfonic acid polymer membrane may be used. In particular, a proton exchange membrane such as NAFION, that conducts protons, may be used for this purpose. Alternatively, anion exchange, bipolar, and ultrafiltration membranes may be used with MFCs, or even no membrane, as in the use of a J-cloth in place of the membrane. Alternatively, the system may comprise a poised potential cell. See Fan et al. (2007).

It is contemplated that any composition, method, or apparatus described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Fermentation of cellulose and hemicellulose to ethanol and electricity by C. thermocellum, T. thermosaccharolyticum and strain Thermincola ferriacetica in an ethanologenic/electricigenic consolidated bioprocess (CBP) at 60° C.

FIG. 2. Sediment fuel cell prepared in an open beaker with graphite anodes in the sediment and overlying water.

FIG. 3. Generation of electric current by thermophilic sediment fuel cells prepared with marine sediment. (FIG. 3A) Current generated by 3 cells incubated at 60° C. (top curves: thick solid line, squares, and triangles) and 3 cells incubated at 22° C. (lower curves: dotted, thin solid, and x lines). (FIG. 3B) Current generated by 3 formaldehyde-killed cells incubated at 60° C. All fuel cells were operated with 1000 Ohm load of resistance. Temporary decreases in current correspond with the replacement of evaporated water.

FIG. 4. Current generated by sediment fuel cells from 22 to 75° C. Maximum sustained current density generated by sediment fuel cells incubated at 22, 45, 60 and 75° C. for 5 days with a 1000-Ohm load resistance. Three cells at each temperature were examined and the error bars represent the standard deviation from the sustained (more than a day) maxima.

FIG. 5. Single-chamber microbial fuel cells with ion exchange membranes (IEM) and air-fed cathodes. Two designs are shown.

FIG. 6. Generation of electricity by thermophilically enriched microbial communities in single chamber fuel cells with Pt-C cloth, air-bathed cathodes and a 1000-Ohm resistance. Three anodes from sediment fuel cells were moved to 3 single chamber cells (dark, gray and thin solid lines) at time zero, were supplied with 25 mM sodium acetate, and were incubated at 60° C. The sediment-free medium and acetate were replaced at each vertical line that meets the x-axis within the plot.

FIG. 7. Cellulose as a fuel for the thermophilically enriched microbial community of Thermincola spp. in a single chamber fuel cell maintained at 60° C. The fuel was switched from acetate to cellulose at the time indicated by the arrow.

FIG. 8. Generation of electricity by Thermincola ferriacetica in a single-chamber fuel cell supplied with acetate and maintained at 60° C. Media and fuel were exchanged from the cell at the arrows while the microbial catalyst remained on the anode as a biofilm.

FIG. 9. An ethanol fermentation vessel with a MFC recycle (an ethanologenic/electricigenic consolidated bioprocess reactor).

FIG. 10. Design of a MFC recycle for use with an ethanol fermentation vessel.

FIG. 11. Components of a MFC recycle.

FIG. 12. An ethanologenic/electricigenic consolidated bioprocess (CBP) bioreactor with a pretreatment MFC.

FIG. 13. Polarization (solid squares) and power curve (open circles) analysis from a single chamber fuel cell incubated at 60° C. and inoculated with Thermincola spp. and Deferribacteres. A variable resistor box was used to set the resistance for each resistive load (150 to 64,000 Ohms) in order to measure the polarization curve at pseudo-steady state.

FIG. 14. Scanning electron micrographs of bacteria on the anode surface of a MFC incubated at 60° C. with acetate as fuel. No biofilm was observed when a MFC was incubated with an open (unconnected) circuit.

FIG. 15. Acetate as a fuel for the thermophilically enriched microbial community in a single chamber fuel cell. Following transfer of an anode from a sediment fuel cell to a single chamber cell without sediment and 6 exchanges of media without sediment, the medium was replaced without acetate. As designated on the plot, acetate was added after the current had dropped by more than 80%, and the electric current was re-established. The cell was operated with a 1000 Ohm resistance.

FIG. 16. An ethanologenic/electricigenic consolidated bioprocess (CBP) using cellulose-containing renewable energy sources such as peach waste and wood fiber.

FIG. 17. Thermophilic anaerobic degradation of cellulose and chitin to electricity and ethanol in a MFC bioreactor.

FIG. 18. Thermophilic BEAMR process.

FIG. 19. Thermophilic hydrogen and ethanol production process.

FIG. 20. Thermophilic hydrogen and ethanol production process where ethanol and hydrogen produced in the same chamber.

FIG. 21. Thermophilic hydrogen production process, which uses another MFC as a power source.

FIG. 22. Thermophilic hydrogen and ethanol production process where ethanol and hydrogen are produced in a single, anaerobic chamber without an electrode-separating membrane.

FIG. 23. MFC apparatus to produce ethanol and electricity where the fermentation takes place in the MFC's anode chamber.

FIG. 24. MFC apparatus to produce ethanol and electricity where the fermentation vessel is separated from the MFC via a permeable membrane.

FIG. 25. An apparatus for generating ethanol and electricity, wherein the microbial fuel cell cathode chamber serves as the fermentation vessel. In addition, this microbial fuel cell has a second pair of cathode/anode. The secondary anode is wired to the secondary cathode, which is an air cathode. The air cathode is located on the other side of the fermentation vessel.

FIG. 26. Generation of ethanol and electricity using the MFC apparatus illustrated in FIG. 23. Cellulose is fuel, the mixed culture containing Thermincola is the electricigen, Clostridium thermocellum is the ethanologen.

FIG. 27. Generation of ethanol and electricity using the MFC apparatus illustrated in FIG. 23. Crushed peach is fuel, the mixed culture containing Thermincola is the electricigen and Clostridium thermocellum is the ethanologen.

FIG. 28. Zymomonas mobilis produced ethanol from crushed peaches in a fermentation vessel under mesophilic conditions. In a separate step, the resulting culture is stripped of ethanol and fed to a fermentation vessel in operable relation with a microbial fuel cell under thermophilic temperatures.

FIGS. 29A-F. Electricity generation from microbial fuel cells inoculated with electricigens and C. thermocellum. (FIG. 29A) A pure culture of T. ferriacetica was used as electricigens, and the fuel cell was inoculated with 5 vol % of C. thermocellum. The result is for one-medium-exchange period. (FIG. 29B) A mixed culture was used as electricigens, and the fuel cell was inoculated with 10 vol % of C. thermocellum. The result is for three-medium-exchange period. (FIG. 29C) A mixed culture was used as electricigens, and the fuel cell was inoculated with 10 vol % of C. thermocellum. The result is for two-medium-exchange period. This experiment is similar to FIG. 29B except that older inoculum of C. thermocellum was used in this case. (FIG. 29D) A mixed culture was used as electricigens, and the fuel cell was inoculated with 5 vol % of C. thermocellum. The result is for one-medium-exchange period. The fuel cell was placed in a sealed plastic bag, and the cell potential was measured manually. (FIG. 29E) A mixed culture was used as electricigens, and the fuel cell was inoculated with 5 vol % of C. thermocellum. The result is for one-medium-exchange period. The fuel cell was placed in a sealed plastic bag, and the cell potential was measured manually. This experiment is similar to FIG. 29D except that a 10000-Ohm resistor was used here. An 1000-Ohm resistor was used for FIGS. 29A-D and FIG. 29F. (FIG. 29F) A typical cell potential profile for a microbial fuel cell inoculated with a mixed culture of electricigens without C. thermocellum where sodium acetate was used as a carbon source, shown for comparison. The result is for three-medium-exchange period. All experiments were performed at 60° C.

FIGS. 30A-E. Ethanol production in the microbial fuel cells inoculated with electricigens and C. thermocellum. (FIGS. 30A-E) These experiments correspond to the experiments in FIGS. 29A-E. In FIGS. 30D-E, the fuel cells were placed in a sealed plastic bag, and the total mass of ethanol produced including the amount permeated through the Nafion® membrane was measured and plotted. The experiments shown in FIGS. 30D-E are very similar except for the resistor used; 1000 Ohm for FIG. 30D and 10000 Ohm for FIG. 30E.

### DESCRIPTION OF ILLUSTRATIVE EMBODIMENTS

### A. The Present Invention

The present invention provides, in one aspect, a process for combining cellulolytic/ethanol fermentation with microbial fuel cell (MFC) technology. A MFC bioreactor comprises fuel cell components, microbial catalysts, and organic compounds that serve as the fuel for electricity generation (Logan et al., 2006). The present process utilizes electricity-generating bacteria, which consume the waste products but not ethanol, as well as cellulolytic ethanol-producing bacteria in MFCs. Thus, the combined system produces higher yields of ethanol while also generating electricity.

In another aspect, the present invention provides a microbial fuel cell that is modified to produce hydrogen. One example of such a system is described in U.S. Patent Publn. 2006/0011491, which discloses a BEAMR system. A BEAMR system is a microbial fuel cell modified to produce hydrogen, includes a power source for addition of a voltage and is distinct from a water electrolyzer.

The current invention provides an alternative method for the production of ethanol, hydrogen, electricity or combinations thereof. Cellulose and hemicellulose are converted into a mixture of ethanol, lactate and acetate and other inhibitory byproducts by anaerobic cellulolytic such as Clostridium spp., while organic acid consuming bacteria remove the inhibiting acids from the fermentation vessel. A microbial catalyst comprising one or more bacteria and having a cellulolytic activity, an ethanologenic activity and an electricigenic activity is therefore required: cellulolytic activity to hydrolyze the cellulose/hemicellulose and convert the resulting sugars into ethanol, and electricigenic (electrode reducing) bacteria to consume the organic acids. This will remove the inhibiting acids from the fermentation vessel and recover the energy lost as waste products. Where electricity is generated, the resulting current can be used to offset the costs of the system, to power sensors and other electronic devices, to generate power for remote applications, and to treat waste. Furthermore, the electricity generated can be used to poise the potential of the fermentation vessel to further enhance ethanol production.

Poising the potential in a fermentation vessel to enhance ethanol fermentation by Clostridium thermocellum has been demonstrated by others. (Shin et al., 2002). In the present invention, if the potential is poised in a microbial fuel cell that is modified to produce hydrogen (e.g., a BEAMR system) with Clostridium thermocellum and a thermophilic microbial catalyst having electricigenic activity and cellulose, ethanol and hydrogen is produced. Furthermore, the ethanol produced by such a system is enhanced due to (i) consumption of the acetate by the electricigens, and (ii) a more optimal redox environment.

The aim of the invention is to enhance bioethanol and/or hydrogen production from plant waste. The current invention results in higher yields of ethanol, hydrogen and/or electricity. Overall, more energy will be extracted from the plant waste (cellulose) for less cost with a better yield than what previously could be done with corn or cellulose. Uses for the invention can include the production of ethanol, including ethanol as a biofuel; degradation of agricultural, municipal, residential, and industrial organic waste; and the generation of electricity or hydrogen.

### B. Microbial Catalysts

One of skill in the art would recognize that the microbial catalyst of the present invention may comprise one, two or three bacteria. For example, the ethanologenic, the cellulolytic, and the electricigenic activities may be performed by a single bacterium. Alternatively, one bacterium may perform one activity and a separate bacterium may perform the remaining two activities. A third alternative would be where there are three separate bacteria each performing one of the desired activities. Table 1 illustrates a variety of bacteria that perform the activities of the present invention.

TABLE 1
Examples of bacteria providing the activities of the current invention

| | | Mesophilic/ | |
|---|---|---|---|
| Bacterium | Accession No. | Thermophilic | Activity(s) |
| Clostridium | ATCC 35319 | Mesophilic | Cellulolytic/ |
| cellulolyticum | | | ethanologenic |
| Clostridium | ATCC 27405 | Thermophilic | Cellulolytic/ |
| thermocellum | | | ethanologenic |
| Thermoanaerobacterium | ATCC 7956 | Thermophulic | Ethanologenic/ |
| thermosaccharlolyticum | | | pentose fermenting |
| Thermoanaerobacterium | DSM 8691 | Thermophilic | Ethanologenic/ |
| saccharolyticum | | | pentose fermenting |
| Saccharomyces cerevisiae | | Mesophilic | Ethanologenic |
| Schizosaccharomyces | | | Ethanologenic |
| pombe | | | |
| Zymomonas mobilis | | Mesophilic | Ethanologenic |
| Pichia stipitis | | | Ethanologenic |
| Candida shehatae | | | Ethanologenic |
| Pachysolen tannophilus | | Mesophilic | Ethanologenic |
| Firmicutes | GenBank EU194835 | Thermophilic | Cellulolytic |
| Firmicutes | GenBank EU194836 | Thermophilic | Cellulolytic |
| Firmicutes | GenBank EU194837 | Thermophilic | Cellulolytic |
| Therminocola sp. | GenBank EU194830 | Thermophilic | Electricigenic |
| Therminocola sp. | GenBank EU194831 | Thermophilic | Electricigenic |
| Therminocola sp. | GenBank EU194832 | Thermophilic | Electricigenic |
| Therminocola sp. | GenBank EU194833 | Thermophilic | Electricigenic |
| Shewanella putrefaciens | | Mesophilic | Electricigenic |
| Geobacter sulfurreducens | | Mesophilic | Electricigenic |
| Thermincola ferriacetica | DSMZ 14005 | Thermophilic | Electricigenic |
| Deferribacteres | GenBank EU194827 | Thermophilic | Electricigenic |
| Deferribacteres | GenBank EU194828 | Thermophilic | Electricigenic |
| Deferribacteres | GenBank EU194829 | Thermophilic | Electricigenic |
| Deferribacteres | GenBank EU194834 | Thermophilic | Electricigenic |

1. Temperature Preferences

Multiple factors limit fermentation of bioproducts, particularly in the production of ethanol from biomass. One factor is the performance and capabilities of biological catalysts in the present invention. The microorganisms may be mesophiles, thermophiles or extreme thermophiles, which are categories according to temperature ranges for growth. Studies have previously been performed with psychrotolerant and mesophilic bacteria that operate from generally 15-30° C. In contrast, thermophilic bacteria are well known for their high metabolic rates and resistance to heat. Many environments reach temperatures above 50° C. either by solar radiation, volcanic activity, industrial processes (waste heat), or by the metabolic action of the microorganisms. Since metabolic rates increase with temperature, microbial generation of electric currents is higher in these environments. The processes of the current invention can be performed at ambient temperatures, but cellulose is hydrolyzed faster and ethanol is more readily harvested by distillation at thermophilic temperatures.

a. Mesophiles

Mesophiles are those microorganisms that grow in the moderate temperature range up to about 45° C., especially those whose optimum growth temperature is 20-40° C. Psychrophiles refer to microorganism whose optimum growth temperature is 20° C. or less. An example of a mesophile for use with the current invention is Clostridium cellulolyticum (ATCC 35319). This organism is a well-studied cellulolytic anaerobe, which produces ethanol, lactate and acetate. Desvaux et al. (2000).

b. Thermophiles

Thermophiles refer to microorganisms whose optimum growth temperature is 50° C. or higher, and more particularly in the range of 50-60° C. Among thermophiles, a microorganism whose optimum growth temperature is 50-70° C. is referred to as a moderate thermophile. Thermophilic bacteria are well known for their high metabolic rates and resistance to heat (Madigan et al., 1999; Madigan et al., 2005).

Thermophilic cellulolytic bacteria are advantageous due to their higher rates of hydrolysis of cellulose and metabolism in general (Demain et al., 2005). In addition, the ethanol product, which also inhibits the fermentation at concentrations above 5%, can be more readily driven off under high temperature (60° C.). Therefore, thermophilic bacteria are of particular use with the current invention. Examples of thermophilic bacteria useful with the current invention include Clostridium thermocellum (ATCC 27405) and Thermanaerobacterium thermosaccharlolyticum (ATCC 7956). Another example includes the thermophilic electricigenic bacterium Thermincola ferriacetica. Sources of moderately thermophilic and low-end hyperthermophilic bacteria include marine and freshwater sediment, municipal and industrial wastewater, compost, and sediment from volcanic springs/vents.

c. Extremophiles

Extremophilic bacteria thrive under extreme conditions of pH, salinity, pressure and temperature. Extreme thermophiles have an optimum growth temperature above 70° C. Bacteria are known to thrive under conditions considered extreme for the growth of plants and animals; from pH 1 to 5 and 9 to 11, in near saturating concentrations of NaCl, from below 0° C. to autoclave temperatures (121° C.), and combinations of all. Microbial life also thrives at temperatures as high as 121° C. (Kashefi and Lovley, 2003). Bacteria that function optimally under extreme conditions may serve as more effective catalysts in microbial fuel cells due to their higher activity, greater stability, longer life, capability of utilizing a broader range of fuels.

2. Activity

The current invention utilizes a variety of bacteria having a variety of activities, in particular cellulolytic activity, ethanologenic activity and electricigenic activity. The combination of these activities increases the effectiveness of the system. For example, C. thermocellum produces cellulases and hemicellulases and converts cellobiose into ethanol and organic acids, but this microorganism does not utilize the pentoses that form during hemicellulose fermentation (reviewed within Demain et al., 2005; Lynd et al., 2002). T. thermosaccharolyticum does not possess cellulases but can convert pentoses into acetate, lactate and ethanol. Production of ethanol from cellulose and hemicellulose at thermophilic temperatures by these two organisms in consortium has been demonstrated (Wang et al., 1983). Electricigenic bacteria operating in a MFC will consume inhibitory end products of cellulose fermentation, including but not limited to lactate and acetate, thereby enabling the continued production of ethanol under thermophilic conditions.

A single bacterium may encompass one or more than one of the desired activities. For example, C. thermocellum and C. cellulolyticum are cellulolytic and ethanologenic. Therminocola ferriacetica, other Thermincola spp. and Deferribacteres are capable of consuming organic acids and generating electricity. Additionally, some Thermincola spp. may be cellulolytic and electricigenic.

a. Cellulolytic Activity

The term “cellulolytic activity” is defined herein as a biological activity which hydrolyzes a cellulosic material (U.S. Pat. No. 7,271,244). In the present invention, cellulose and hemicellulose are converted into a mixture of ethanol, lactate and acetate by a microorganism having cellulolytic activity. An example of a mesophilic bacterium that has cellulolytic activity is Clostridium cellulolyticum. This organism is a well-studied cellulolytic anaerobe, which produces ethanol, lactate and acetate (Desvaux et al., 2000). An example of a thermophilic bacteria that has cellulolytic activity is Clostridium thermocellum (ATCC 27405). This bacteria in consortium with Thermoanaerobacterium thermosaccharolyticum (ATCC 7956) converts cellulose and hemicellulose to ethanol plus acetate and lactate. Several other strains of these species are available in commercial culture collections. Another species of Thermoanaerobacterium (T. saccharolyticum DSM 8691), capable of consuming pentoses, is also available. See Table 1, supra, for additional examples. See Lynd et al. (2002), incorporated herein by reference, for further examples.

Examples of fungi useful with the current invention as a microbial catalyst include those of the generas Myrothecium, Chaetomium, Trichoderma, Memnoniella.

b. Ethanologenic Activity

Ethanologenic microorganisms are known in the art and include ethanologenic bacteria and yeast. The term “ethanologenic” is defined as the ability of a microorganism to produce ethanol from a carbohydrate as a primary fermentation product. The term includes naturally occurring ethanologenic organisms, organisms with naturally occurring or induced mutations, and organisms which have been genetically modified. U.S. Pat. Pub. 2002/0137154.

It is well known, for example, that Saccharomyces (such as S. cerevisiae) are employed in the conversion of glucose to ethanol. Other microorganisms that convert sugars to ethanol include species of Schizosaccharomyces (such as S. pombe), Zymomonas (including Z. mobilis), Pichia (P. stipitis), Candida (C. shehatae) and Pachysolen (P. tannophilus), U.S. Patent Publn. 2003/0054500. Additional examples of ethanologenic microorganisms include ethanologenic microorganisms expressing alcohol dehydrogenase and pyruvate decarboxylase, such as can be obtained with or from Zymomonas mobilis (see U.S. Pat. Nos. 5,000,000; 5,028,539; 5,424,202; and 5,482,846, U.S. Patent Publn. 2003/0054500, all of which are incorporated herein by reference). See Table 1, supra, for additional examples.

c. Electricigenic Activity

An electricigenic microorganism is any microorganism that will generate electricity without the addition of a mediator. Not to be limited to one theory, the microbial catalyst having an electricigenic activity may catalyze an electrode reduction in a MFC by reducing a soluble mediator that they produce themselves (Bond et al., 2005; Rabaey et al., 2005; Rabaey et al., 2004), or by reducing the electrode through direct contact. Shewanella putrefaciens (Kim et al., 2002) and Geobacter sulfurreducens (Bond et al., 2003) are non-limiting examples of mesobiotic electricigens. Both are Gram-negative bacteria that are capable of reducing insoluble metal oxides external to the cell, a feature common to electricigens. See Lovley (2006).

Thus far, most of the electricigenic bacteria discovered have been mesophilic. However, thermophilic electrode reduction has been reported above 50° C. In one case (Choi et al., 2004), thermophilic Bacillus spp. were shown to generate an electric current, but only when a soluble synthetic electron-carrying mediator (azure A) was added to the cell (Jong et al., 2006) reported that electricity could be generated with wastewater in a fuel cell incubated at 50° C.

The inventors demonstrated that a thermophilic electricity generating community derived from marine sediment and fueled with acetate consisted of Therminocola spp. and Deferribacteres (Table 2) (Mathis et al., unpublished).

TABLE 2
Analysis of 16S rRNA genes from acetate fueled thermophilic MFC

| | GenBank | | | |
|---|---|---|---|---|
| | Accession | | | % |
| RFLP | No. | Phylum (>90%) | Closest Match (Accession No.) | Similarity |
| A(4)* | EU194828 | Deferribacteres | Uncultured bacterium clone 165B42 | 87 |
| | | | (DQ925879.1) | |
| B(48) | EU194830 | Firmicutes | Thermincola carboxydiphila strain 2204 | 99 |
| | | | (AY603000.2) | |
| C(1) | EU194829 | Deferribacteres | Uncultured bacterium clone C74 | 96 |
| | | | (DQ424926.1) | |
| D(6) | EU194834 | Deferribacteres | Uncultured bacterium clone 1A162 | 89 |
| | | | (DQ424915.1) | |
| E(6) | EUI94831 | Firmicutes | Thermincola carboxydiphila strain 2204 | 99 |
| | | | (AY603000.2) | |
| F(4) | EU194832 | Firmicutes | Thermincola carboxydiphila strain 2204 | 90 |
| | | | (AY603000.2) | |
| G(3) | EU194835 | Firmicutes | Uncultured bacterium clone TTA_B61 | 98 |
| | | | (AY297976.1) | |
| H(1) | EU194833 | Firmicutes | Thermincola carboxydiphila strain 2204 | 88 |
| | | | (AY603000.2) | |
| I(1) | EU194837 | Firmicutes | Uncultured low G + C Gram-positive | 92 |
| | | | bacterium clone | |
| | | | DR546BH1103001SAD28 (DQ234647.1) | |
| J(5) | EU194836 | Firmicutes | Uncultured soil bacterium clone UE5 | 89 |
| | | | (DQ248237.1) | |
| K(1) | EU194827 | Deferribacteres | Uncultured bacterium clone 165B42 | 87 |
| | | | (DQ925879.1) | |

*RFLP pattern (no. of clones)

The inventors have also documented operation of sediment MFCs at thermophilic temperatures and have identified the first thermophilic electricigen, Thermincola ferriacetica (DSMZ 14005). This organism is unique as an electricigen in that it is a thermophile, is Gram positive, and produces autoclave resistant spores. It does not require the addition of a soluble mediator to transfer the electrons to the electrode. They have also demonstrated that a group of Thermincola spp. (16S RNA sequences listed under GenBank Accession Nos. EU194830, EU194831, EU194832, EU194833) most related to Thermincola ferriacetica (88 to 99% similarity by 16S rRNA gene sequence) will generate electricity with acetate or cellulose as a fuel. These bacteria are also Gram positive thermophiles that produce autoclave resistant spores and do not require the addition of a soluble mediator to transfer electrons to an electrode.

3. Sources of Bacteria

MFCs operated at mesophilic temperatures (below 50° C.) have produced power from the oxidation of fuels in ocean sediments (Holmes et al., 2004; Reimers et al., 2001; Tender et al., 2002), wastewater (Angenent et al., 2004; Logan, 2005; Min et al., 2005) and biomass (Wilkinson, 2000). Temperatures above 50° C., produced by direct sunlight, volcanic hot springs, hydrothermal vents, composting of municipal and agricultural waste, steam lines and hot water pipes, and the waste heat from a variety of industrial processes, are supportive of the growth of thermophilic bacteria (Madigan et al., 1999; Madigan et al., 2005). Soil and aquatic sediments from temperate environments are known to possess thermophilic bacteria with optimal growth temperatures above 50° C. (Madigan et al., 2005). Sediments are also rich with many of the recently discovered mesophilic, direct electrode-reducing bacteria (Bond et al., 2002; Holmes et al., 2004). Multiple means of isolation are known to those skilled in the art. Alternatively, many such bacteria are deposited. See Table 1 for ATCC citations.

The community of Thermincola spp. enriched from marine sediment by the inventors was initially enriched on an electrode of a MFC supplied with acetate as fuel. The culture was further enriched by 1) repeated serial transfer from MFC to culture media with acetate and insoluble iron, 2) autoclaving for 30 minutes, 3) cultivation in a MFC with acetate as fuel, 4) isolation as a colony on agar containing media with acetate and fumarate, 5) autoclaving again, 6) cultivation in a MFC and again in acetate plus insoluble iron media. After this enrichment/isolation procedure, the culture would use acetate or cellulose as fuel and generate electricity in a MFC. The culture contained only Gram positive rods that produce autoclave resistant spores. Before autoclaving, the community consisted of Thermincola spp. and Deferribacteres (Table 2). After autoclaving, only Thermincola spp. remained. The 16S RNA sequences of the Thermincola spp. are found under GenBank Accession Nos. EU194830, EU194831, EU194832, EU194833.

### C. Fuel Sources

The energy to be harvested is also dependent upon the fuel. Biomass or organic waste will commonly include cellulose, which is the most abundant carbon source on the planet and an excellent potential renewable energy source. For a review of cellulose and its fermentation see Demain et al. (2005). Electricity generation by MFCs supplied with cellulose has been reported with mesophilic bacterial catalysts (Rismani-Yazdi et al., 2007; Ren et al., 2007). It has been demonstrated that hydrogen generated by mesophilic cellulolytic bacteria can be collected and then abiotically transformed into electricity by a fuel cell (Niessen et al., 2005).

The fuel source may be any biomass or organic waste that may be consumed to generate ethanol, hydrogen or electricity. Examples of a fuel sources for use with the current invention include acetate, sugars, cellulose, hemicellulose or chitin. Potential sources of cellulose may include corn stover, peach waste, plant residues, forest litter, chitin and switchgrass. Examples of suitable plant residues include stems, leaves, hulls, husks, cobs and the like, as well as wood, wood chips, wood pulp and sawdust.

In one embodiment, a cellulolytic and electricigenic microbial catalyst will consume cellulose and generate electricity and acetate. This acetate can be further consumed by an electricigenic microbial catalyst and additional electricity is produced. In a further embodiment, an ethanologenic microbial catalyst will consume cellulose to produce ethanol and acetate. This acetate can again be further consumed by an electricigenic microbial catalyst to produce electricity. As a non-limiting example, Clostridium thermocellum may be combined with celluose to produce ethanol and acetate. T. ferriacetica may additionally be added to consume the acetate and generate electricity.

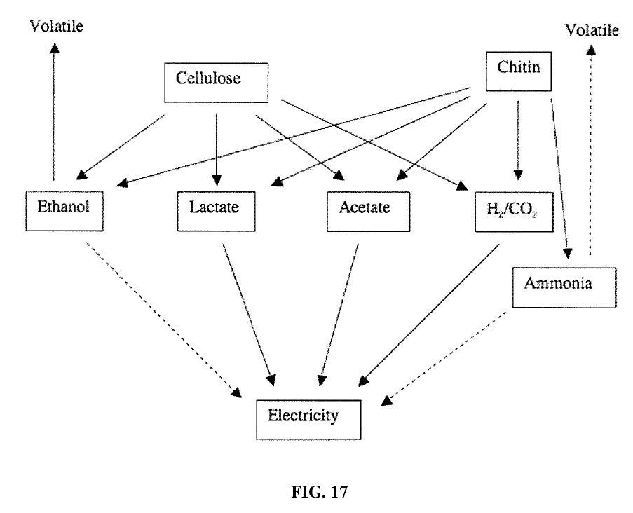

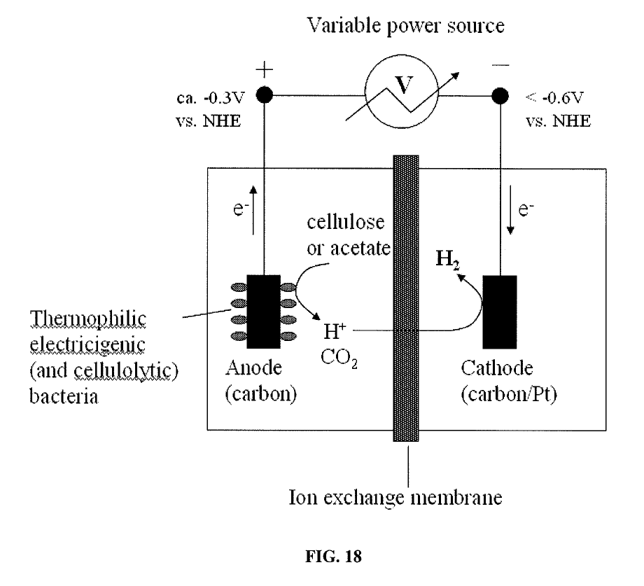

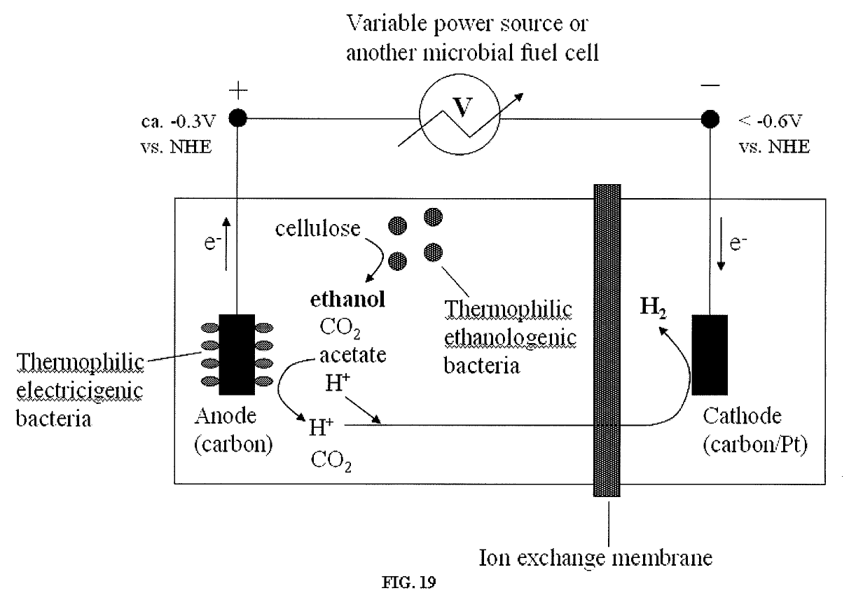

The microbial catalysts having cellulolytic activity of the current invention are also capable of degrading chitin. Chitin is one the most prevalent biopolymers on the planet. Sources of chitin include insects, fungi, crustaceans and diatoms. FIG. 17 demonstrates how chitin and cellulose may be degraded by a microbial catalyst having cellulolytic activity.

In some processes of the current invention, acetate is produced as a byproduct. This acetate may be consumed by an electricigenic microbial catalyst to generate electricity. For example, acetate can serve as a fuel for electricity generation by thermophilic bacterial communities enriched from marine sediment or by Thermincola ferriacetica. T. ferriacetica is capable of using acetate as a sole carbon and energy source for growth with insoluble iron as an electron acceptor (Zararzina et al., 2007). Furthermore, T. ferriacetica will generate electricity in a fuel cell when using acetate as a fuel. Acetate will also serve as a fuel for the community of Thermincola spp. that the inventors enriched from marine sediment.

### D. Bioprocesses

1. Cellulosic Ethanol Fermentation

When lignocellulose serves as the raw material, it is usually pretreated to render the cellulose and hemicellulose fractions more accessible to cellulases and hemicellulases. The pretreatment generally consists of a dilute acid treatment under high temperature (Schell et al., 2003). Lignin is separated and used to fire boilers for the production of steam that is used to drive electric generators, and the cellulose/hemicellulose slurry must be neutralized for pH and cooled before transfer to the fermentation vessel (Aden et al., 2002).

Two microbial processes in particular are useful as a way to ferment cellulose and hemicellulose into ethanol: simultaneous saccharification and co-fermentation (SSCF) and consolidated bioprocessing (CBP).

a. Simultaneous Saccharification and Co-Fermentation

In one embodiment, the invention provides a process of generating ethanol and electricity comprising supplying a microbial catalyst having ethanologenic activity and a fuel source to a first fermentation vessel, wherein a spent fuel source is generated, supplying the spent fuel source and a microbial catalyst having a cellulolytic activity and an electricigenic activity to a second fermentation vessel, wherein the second fermentation vessel is in operable relation with a microbial fuel cell and wherein the spent fuel source is consumed and ethanol and electricity are generated. This may be achieved by the process of simultaneous saccharification and co-fermentation. See Takagi et al. (1977).

In a simultaneous saccharification with co-fermentation of hexose and pentose sugars (SSCF) system, cellulases are prepared in a separate step and Zymomonas mobilis (or similar organism) is used to ferment the sugars to ethanol. (U.S. Pat. No. 3,990,944 and U.S. Pat. No. 3,990,945). SSCF requires the production of cellulases in a separate process. These cellulases are frequently thermophilic enzymes that are incubated with the pretreated cellulose to produce sugars. The sugars are then fermented in a second step once the temperature has been lowered. Zymomonas mobilis, Saccharomyces cerevisiae, Escherichia coli and Klebsiella oxytoca or engineered strains of these are then used to convert the sugars into ethanol.

b. Consolidated Bioprocess (CBP)

In contrast to processes featuring a step dedicated to the production of cellulase enzymes, the cellulose and hemicellulose may alternatively be fermented by consolidated bioprocessing (CBP), which combines cellulase production, cellulose hydrolysis and fermentation into one step (Lynd et al., 2005). It has been estimated that CBP decreases capitol and operating costs by more than 4-fold (Lynd et al., 2005).

CBP is approached by either the genetic introduction of cellulases into non-cellulolytic bacteria or through the use of cellulolytic anaerobic bacteria. The latter may be achieved with combinations of cellulolytic thermophiles that complement the metabolism of the other. For example, the combination of Clostridium thermocellum and Thermoanaerobacterium thermosaccharolyticum thermophiles is advantageous due to their high metabolic rates and because they complement the metabolism of one another. In particular, C. thermocellum produces some of the fastest and most effective cellulases and hemicellulases known, but it will not consume the pentoses produced from hemicellulose. T. thermosaccharolyticum will consume the pentoses and both organisms produce a mixture of ethanol, lactate and acetate. Hydrogen and CO₂ are also formed, but ethanol and acetate are the primary products.

2. Production of Ethanol and Hydrogen

In one embodiment, the current invention provides a process of generating hydrogen comprising supplying a microbial catalyst and a fuel source to a BEAMR system under thermophilic conditions, wherein the microbial catalyst consumes the fuel source and generates hydrogen. Broadly described, the system comprises a fuel source that is oxidized by thermophilic electricigenic bacteria that generate electrons and protons. A power source is connected to the microbial fuel cell to provide an additional voltage. The electrons generated by the bacteria are transferred to the anode, and, through a conductive connector, to the cathode. Oxygen is substantially excluded from the cathode area such that protons and electrons combine at the cathode, producing hydrogen. U.S. Ser. No. 11/180,454. This process is illustrated in FIG. 18.

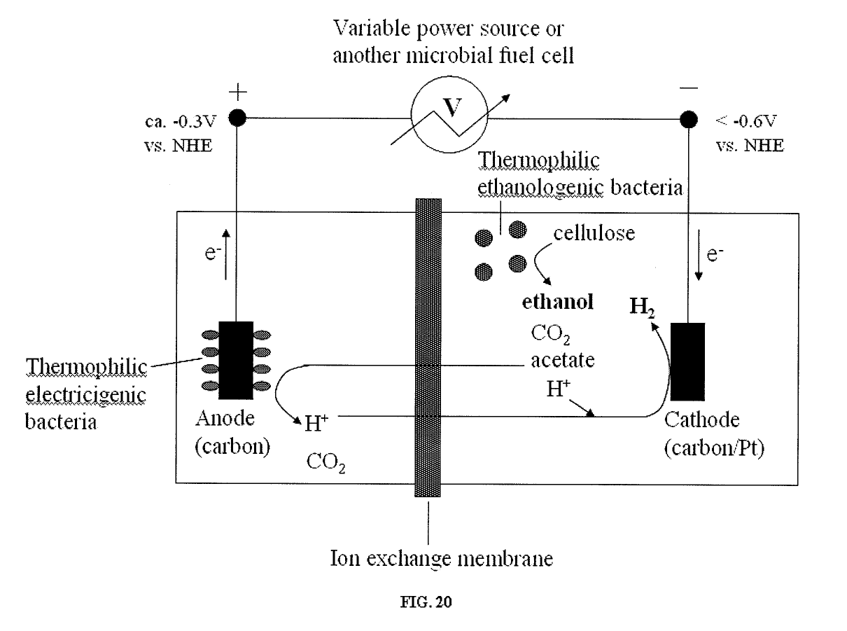

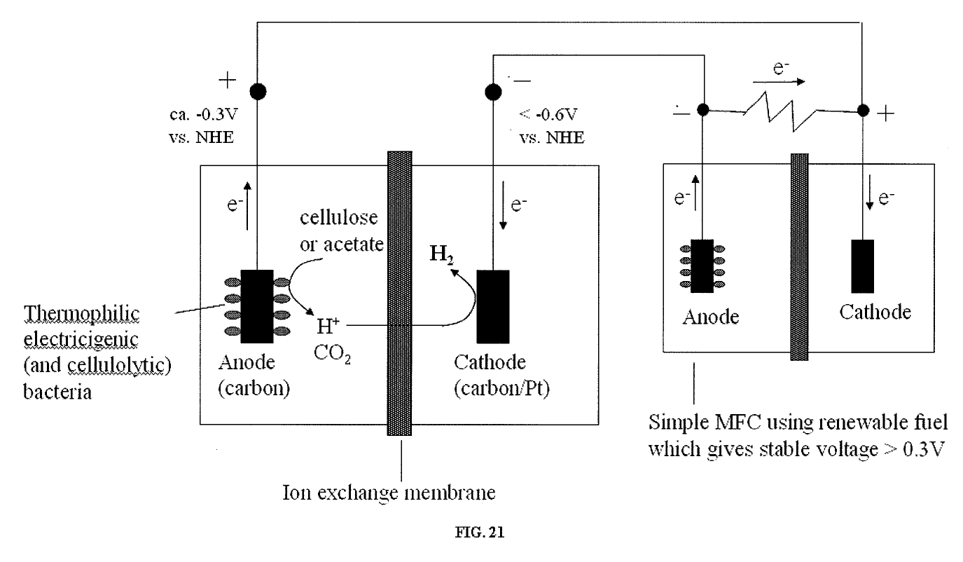

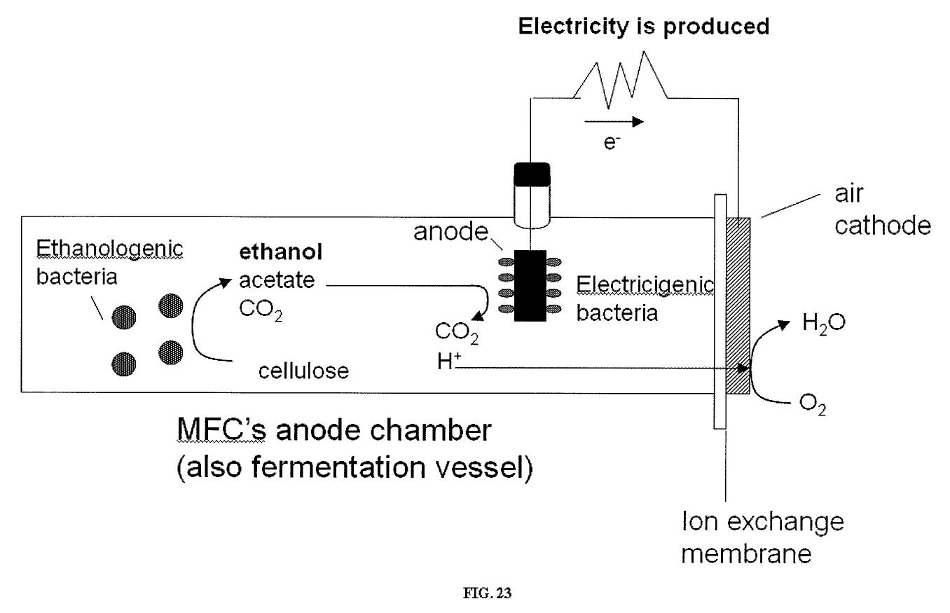

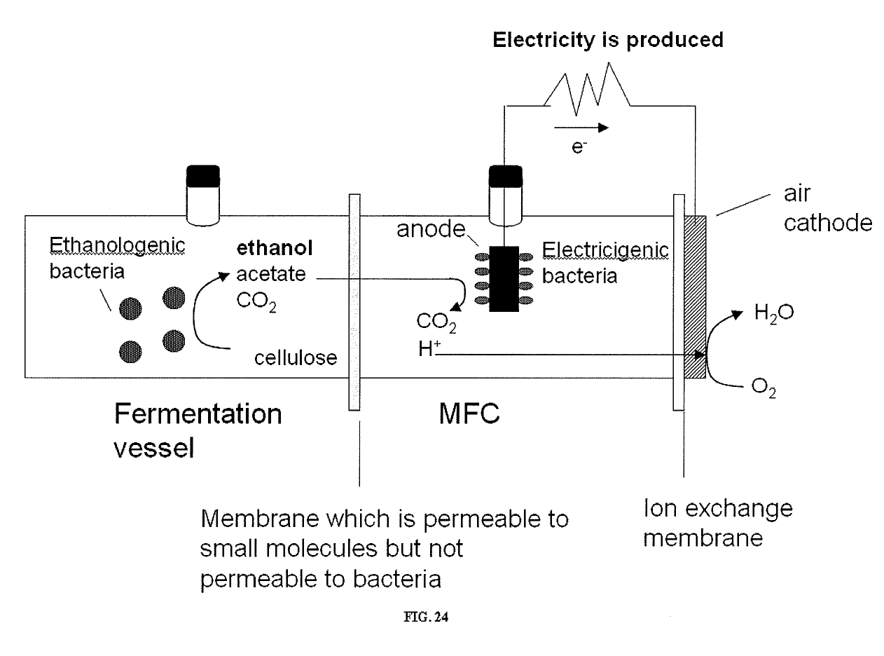

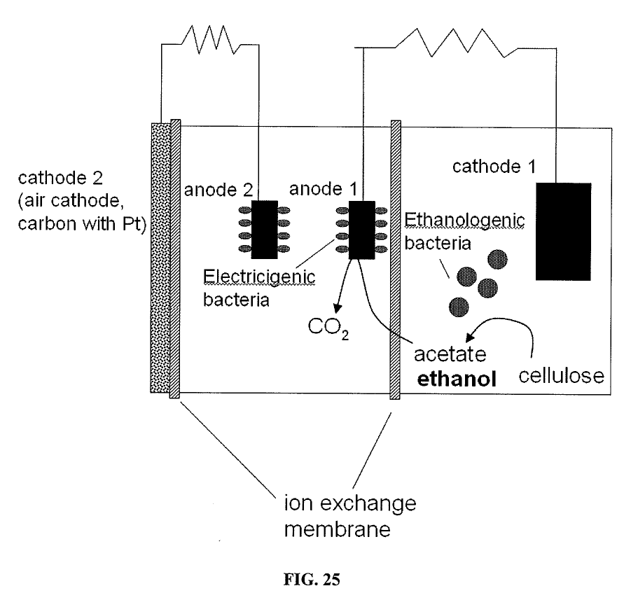

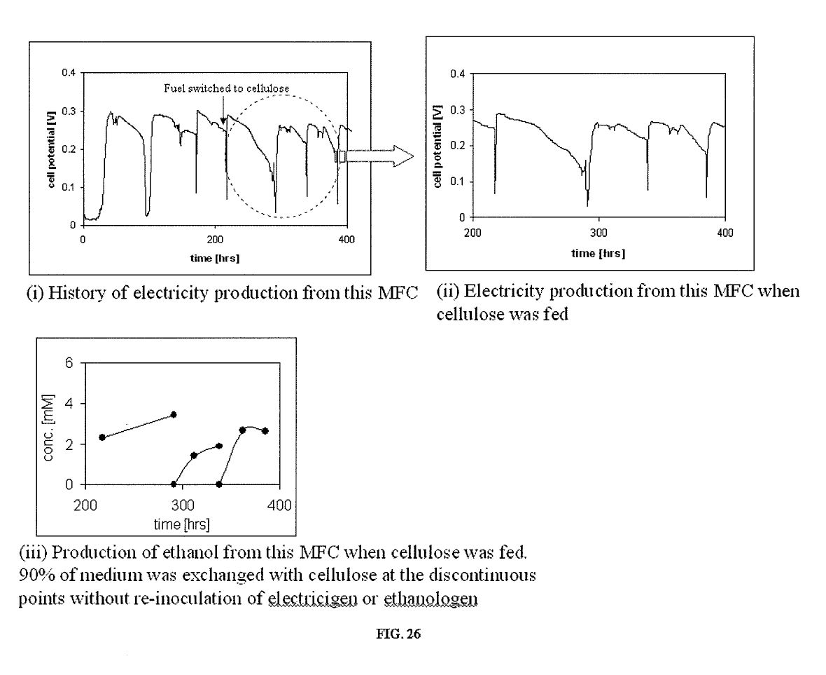

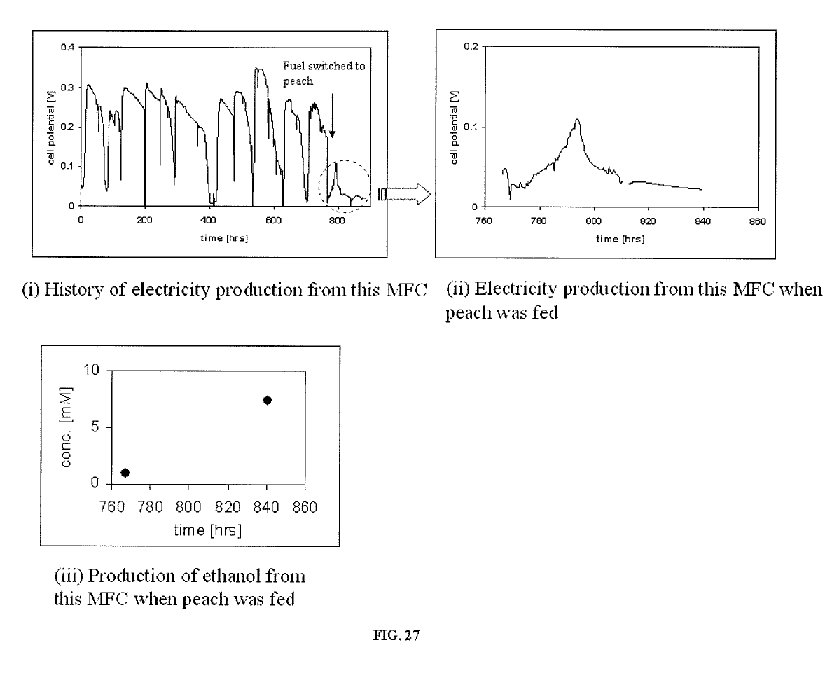

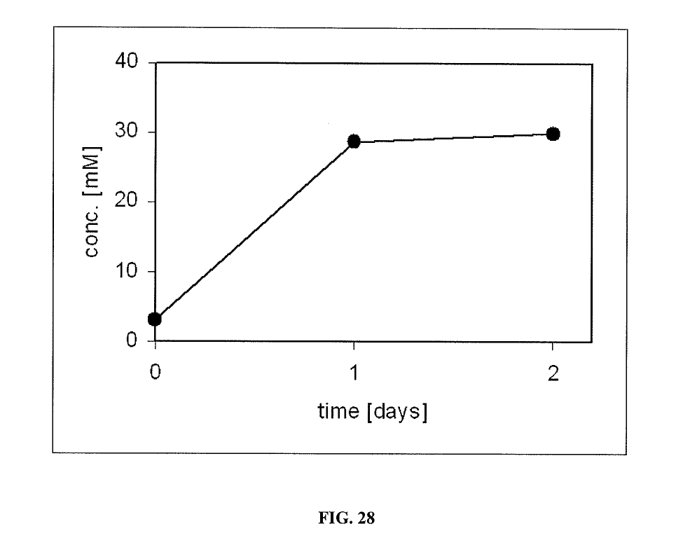

In a further embodiment, the invention comprises a process of generating ethanol and hydrogen comprising supplying a microbial catalyst and a fuel source to a BEAMR system, wherein the microbial catalyst has a cellulolytic activity, an ethanologenic activity, and an electricigenic activity, wherein the fuel source is consumed and ethanol and hydrogen are generated. The microbial catalyst may comprise one or more bacteria. Various modifications may be made to the process. For example, the ethanol and hydrogen may be produced in the same chamber or in separate chambers. FIG. 20 illustrates a process where the ethanologenic bacteria are supplied to the anode side of a system where a membrane separates the anode and the cathode. Alternatively, FIG. 21 illustrates a process where ethanol and hydrogen are produced in the same chamber. This is achieved by supplying the ethanologenic bacteria to the cathode side of a single chamber system where a membrane separates the anode and the cathode. Another variation is demonstrated in FIG. 23, where the hydrogen and the ethanol are produced in a single, anaerobic chamber with no membrane separating the anode and the cathode.

### E. Apparati

1. Microbial Fuel Cells

A microbial fuel cell (MFC) refers to a device that uses bacteria as catalysts to oxidize a fuel source and generate electrons that are transferred to an anode. The generation of electricity by bacteria has been explored since at least 1910 when Potter constructed and analyzed the operation of what could be described as early versions of MFCs (Potter, 1910; Potter, 1912). The pace of discovery in this field is increasing, and today there is a growing interest in the discovery of new and environmentally sound energy technologies. Power densities (per electrode surface area) have exceeded 1 W/m² in recent research with oxygen-supplied cathodes (Jong et al., Environ. Sci. Technol. on-line, 2006; Liu et al., 2005). This is enough electricity to power microelectronic devices or at a large scale to power lighting, charge batteries, operate small pumps, or in the case of bioethanol production, reduce the utility costs of the operation. For an up to date review on the methodology of MFCs, see Logan et al. (2006).

The basic design of a MFC comprises an anode connected to a cathode with a fixed external resistance placed in line. The anode is maintained in an anoxic environment while the cathode is exposed to an oxidizing agent, such as ferricyanide or more commonly oxygen. The anode may be comprised of any material that allows oxidation. See Rosenbaum et al. (2007); Qiao et al. (2007). The cathode may be comprised of any material that allows reduction. Regardless of the design of the MFC, anoxic conditions within the anode chamber favor electricity production by sustaining anaerobic growth and metabolism and also avoiding microbial and abiotic consumption of the fuels.

One of the simplest designs is the placement of the anode into anaerobic sediment and connecting it to a cathode in the overlying oxygenated water (Reimers et al., 2001; Tender et al., 2002). Electrons released during biological consumption of reduced organic and inorganic compounds travel the wire while a current of protons migrate from the anode to the cathode via the sediment and water. In the absence of sediment, an ion exchange membrane (IEM) that is relatively impermeable to oxygen is usually used to separate the anode from the cathode. IEMs have been used to construct dual and single chamber MFCs; in the latter case the IEM is often fused to a cathode bathed in air on one side (Liu et al., 2004; Liu et al., 2004). A higher voltage can be produced, at least temporarily, in the absence of an IEM (Liu et al., 2004), but this allows more oxygen into the anode chamber, which can reduce the efficiency of the MFC.

Power output of a MFC can also be enhanced by the application of a catalyst to the cathode to facilitate the reduction of oxygen. The cathode catalyst may be comprised of any material that increases the rate of the reaction. For example, the cathode catalyst may be comprised of platinum or lead dioxide. See Yu et al. (2007) and Morris et al. (2007). Non-platinum cathode catalysts may also be used (Yu et al., 2007). For example, iron(II) phthalocyanine and cobalt tetramethoxyphenylporphyrin have recently been shown to serve nearly as well (Cheng et al., 2006; Zhao et al., 2005) and are far less expensive.

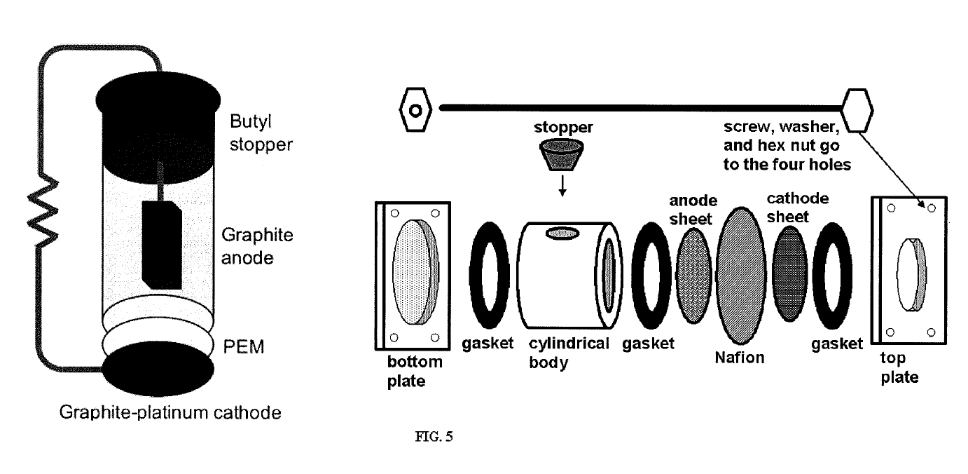

In one embodiment, the single chamber fuel cell has a graphite anode block within and an air-bathed cathode of cloth carbon-Pt (0.5 mg Pt/cm²) covering one end of the cell (FIG. 5), which enhances availability of oxygen to the cathode, thereby increasing overall electron transfer from the bacteria to electrodes. A proton (cation) exchange membrane juxtaposed to the inside of the cathode allows for the passage of protons from the anode to the cathode, prevents the entrance of oxygen into the cell, and slows evaporation. Medium, bacterial cells, and fuel can be delivered through a butyl stopper fitted at the end opposite to the cathode. The entire apparatus may be maintained at thermophilic temperatures. In the alternative, an alternative fuel cell design with a flat cloth anode that may be positioned near or far from the ion exchange membrane and cathode (FIG. 5) may alternatively be used.

In a MFC, bacteria use an anode as a terminal electron acceptor. It is known that some species of bacteria are capable of donating electrons to an electrode within a MFC. (Lovley, 2006). The electrons are released during the consumption of an organic compound (e.g., acetate) and an electric current is generated. Alternatively, the bacteria may use a soluble factor from the environment as an electron carrier to mediate transfer of electrons to the electrode, require the addition of a synthetic mediator (Park et al., 2000), generate a soluble mediator (Rabaey et al., 2005; Rabaey et al., 2004), or through direct bacterium-to-electrode contact deliver electrons to the surface of an electrode (Lovley, 2006).

2. Ethanol Fermentation Plus Microbial Fuel Cells

In one embodiment, the process produces ethanol and electricity from plant waste material. The operation of this system requires the combination of an ethanol fermentation process with a microbial fuel cell (MFC). MFCs are used to make electricity with bacteria as the catalysts. In one embodiment, a MFC recycle attached to a stirred tank reactor (FIG. 9). The present invention is designed to operate in batch, semi-batch or with a continuous feed of biomass and nutrients at 60° C. Cellulose or pretreated biomass slurry is supplied to the fermentation vessel along with nutrients required for the growth of the microbial catalyst.

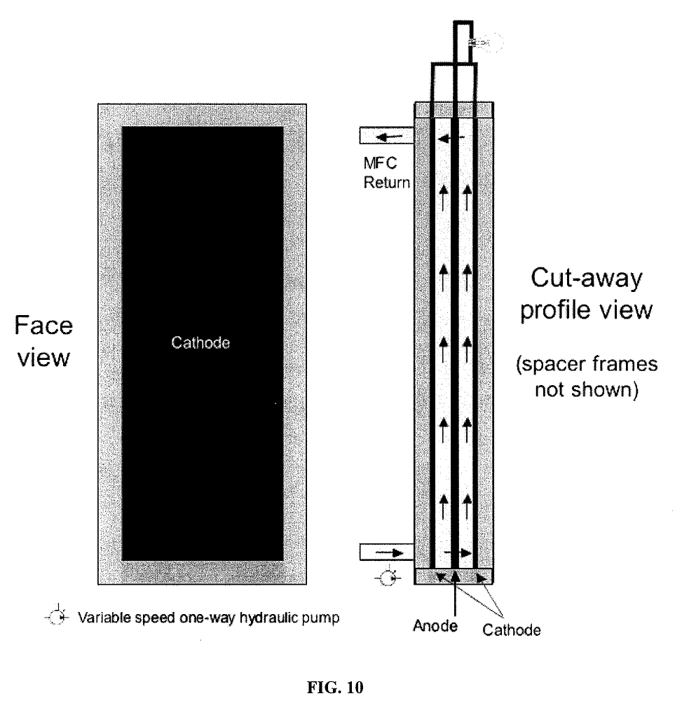

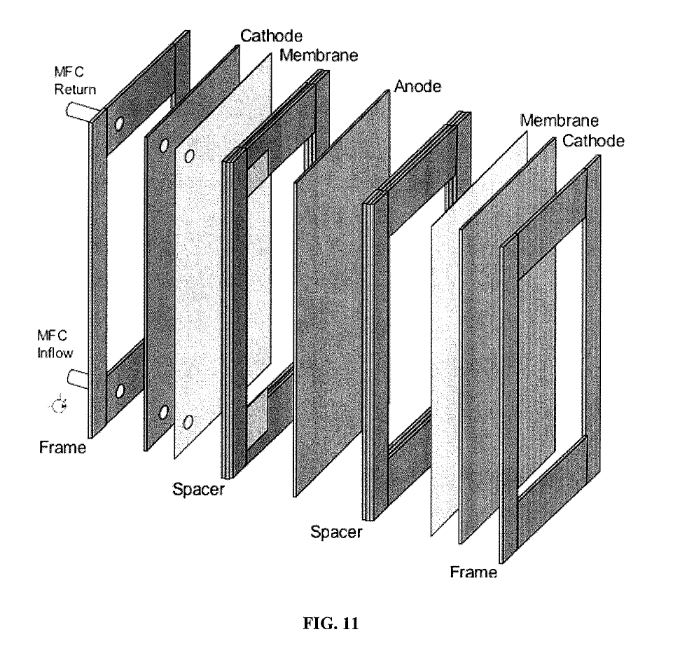

At the bottom of the fermentation vessel an inflow valve will lead to a flat plate MFC. The MFC may comprise a large plate of graphite as anode that runs through the middle of the MFC (FIGS. 10 and 11). This presents the electricigens with a plentiful supply of surface area and electron acceptor. Reaction at the cathode is often a limiting factor in the operation of a MFC (reviewed in Logan et al., 2006). In some embodiments two air-bathed cathodes, one each on the outer surface of the MFC may be employed. The anode is connected to each of the cathodes through a resistive load and the voltage, current and power determined as described above for work with the sediment and single-chamber MFCs. A cation exchange membrane (e.g., Nafion) may coat the inside surface of each carbon cloth cathode. One of skill in the art would be familiar with a variety of options for a membrane. See Fan et al. (2007). In some embodiments, the addition of a catalyst to the cathode also enhances cathode performance, for example Platinum. Other alternative approaches to the design include the use of cobalt tetraporphyrin in the cathode, which has been shown to be nearly as effective as platinum at catalyzing the reduction of oxygen in MFCs (Cheng et al., 2006; Zhao et al., 2005). Innovative microbially catalyzed cathode reactions have also been recently demonstrated in the literature (ter Heijne et al. 2006).

The structure and size of the MFC can also be altered to improve the CBP. For example, the MFC can be designed with a serpentine flow system, in order to enhance structural support (see Min et al., 2004). Graphite carbon can be used in the anodes and cathodes. Alternatively, less expensive carbon fibers may be used to reduce the cost of the electrodes. Higher power densities have been achieved without an IEM in the MFC. (Liu et al., 2004). The increased surface area of the cathode and anode substantially increases the capacity of the MFC to consume the organic acids that inhibit the ethanol fermentation in the vessel. Much of the ethanol is stripped from the fermentation broth before entering the MFC. A gas sparger (N₂) may be added to the MFC inflow line to additionally strip ethanol away from the fermentation broth, which helps prevent inhibition of the consumption of the organic acids by the electricigens.

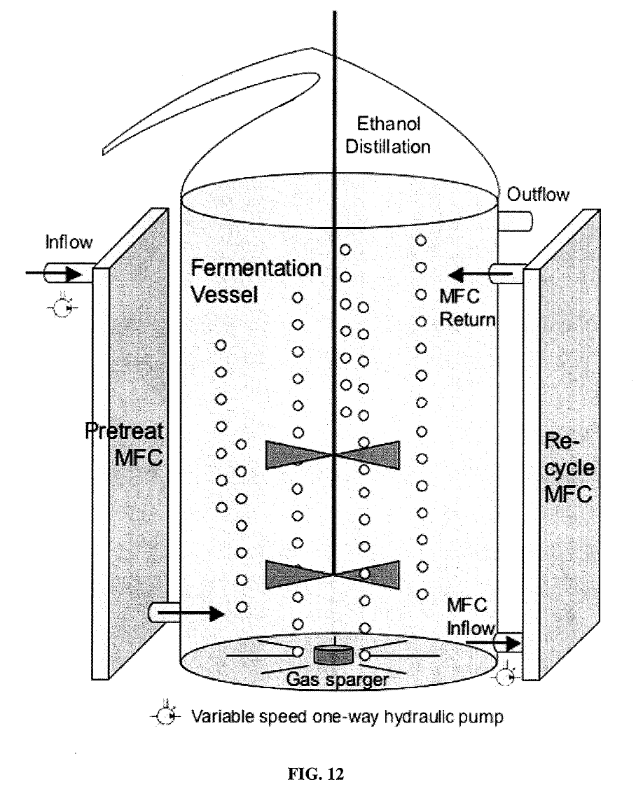

A MFC with electricigenic bacteria, thermophilic or mesophilic, can also be used to further treat the pre-treated lignocellulose before application to the ethanologenic process. In this way, the inhibitory acetate produced during the pretreatment could be consumed before introduction of the biomass into a CBP, or into a SSCF. A combination of a pretreatment MFC coupled to an ethanologenic fermentor with a recycle MFC is presented in FIG. 12. For example, an alternative to combining T. ferriacetica with C. thermocellum and T. thermosaccharolyticum in an ethanologenic/electricigenic CBP would be to enrich for thermophilic electricigens in the presence of the cellulolytic and fermentative organisms and pretreated biomass (e.g., pretreated corn stover).

3. Hydrogen Production

A microbial fuel cell may be modified to produce hydrogen. An example of such a microbial fuel cell is a bio-electrochemically assisted microbial reactor (BEAMR). Such a system is described in U.S. patent application Ser. No. 11/180,454. Broadly described, the system comprises a fuel source that is oxidized by bacteria that generate electrons and protons. A power source is connected to the microbial fuel cell to provide an additional voltage. The electrons generated by the bacteria are transferred to the anode, and, through a conductive connector, to the cathode. Oxygen is substantially excluded from the cathode area such that protons and electrons combine at the cathode, producing hydrogen. The system may be maintained under thermophilic conditions.

Fuel sources oxidizable by electricigenic bacteria are known in the art. Illustrative examples include, but are not limited to, acetate, sugars, cellulose, hemicellulose or chitin. Potential sources of cellulose may include corn stover, peach waste, plant residues, forest litter, chitin and switchgrass. Examples of suitable plant residues include stems, leaves, hulls, husks, cobs and the like, as well as wood, wood chips, wood pulp and sawdust.

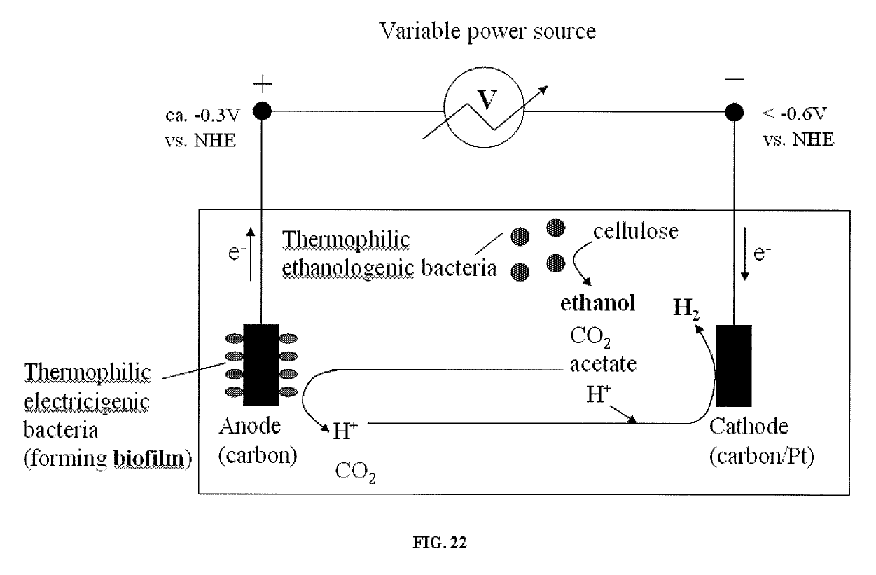

Power sources used for enhancing an electrical potential between the anode and cathode are not limited and illustratively include grid current, solar power sources, wind power sources. Further examples of a power source include a DC power source and an electrochemical cell such as a battery or capacitor. Alternatively, a second MFC may be used as the power source (see FIG. 22).

An ion exchange membrane (IEM) that is relatively impermeable to oxygen may be used to separate the anode from the cathode. IEMs have been used to construct dual and single chamber systems. One of skill in the art would be familiar with a variety of options for a membrane. See Fan et al. (2007). Alternatively, a single, anaerobic chamber without membrane may be utilized (see FIG. 23).

### F. Applications

The present invention may be useful for a variety of applications. One example is the use of the invention for bioprocessing of peach, wood waste, corn stover, switchgrass or any other cellulose-based waste or fuel into ethanol and electricity. The fundamental process described above can be leveraged to generate ethanol and electricity from waste streams common to agriculture in some regions: A second example is solely the generation of electricity from such cellulose-based fuels. A third example is the generation of ethanol and hydrogen from such cellulose-based fuels. And a fourth example is solely the generation of hydrogen from cellulose-based fuels. Each of these examples would utilize the thermophilic microbial catalysts described in a modification of a microbial fuel cell.

Use of this technology may create a significant increase in the number of jobs associated with the operation of bioethanol plants in regions that does not have ready access to current ethanol fuel sources but could use this renewable energy technology. Additionally, corn ethanol does not displace very much fossil fuel (a few percent at best), but it is estimated that cellulosic ethanol could replace 30 to 40% of petroleum required for transportation. Ethanol production from corn stover, waste paper, peach waste, and wood chips clearly would contribute. The same can be said if the process is altered to produce hydrogen. In the former case a liquid transportation fuel is being produced, in the latter case a fuel for hydrogen fuel cells, stationary or for transportation, is being produced. The process using the biocatalysts described can also be modified to produce solely electricity, which could be used to off-set local energy needs. In the long term, the results from this project will help various regions become more energy-independent by producing renewable energy and fuel. This will cut expenditure for importing fuel and saving money. If the facilities to convert the wastes to ethanol and energy are built, hundreds of jobs will be created in that location.

### G. Examples

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Enrichment and Isolation of Thermophilic Microorganisms from Marine Sediment

1. Methods

a. Sediment Fuel Cells

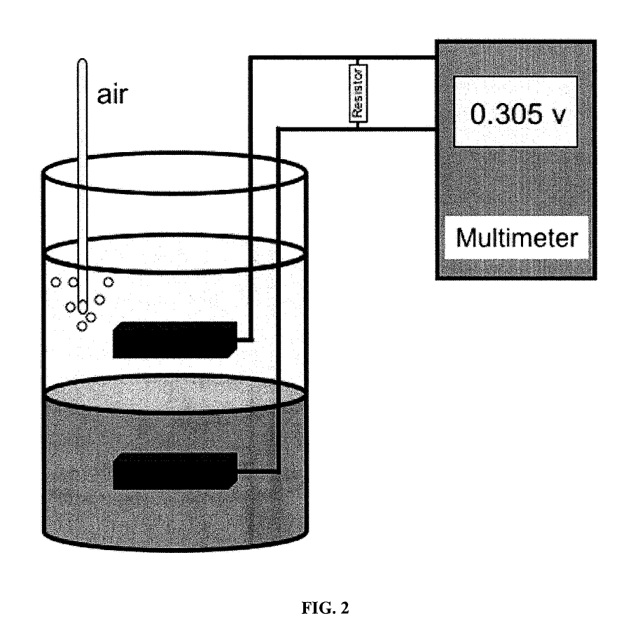

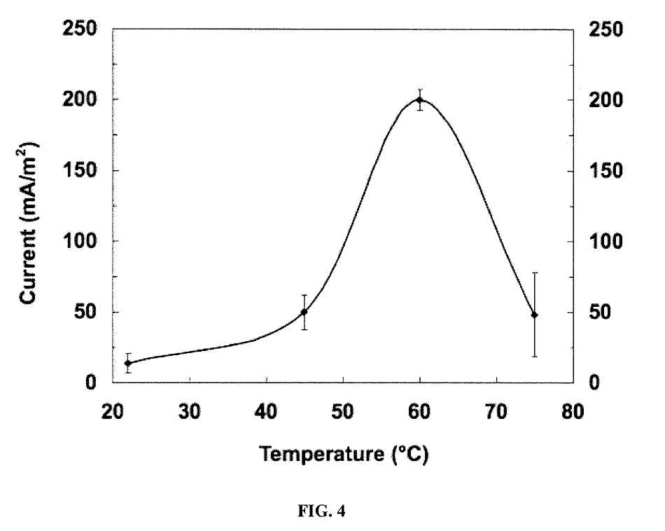

Anoxic marine marsh sediment 2 to 30 cm below the sediment surface was collected along the banks of the mouth of the Ashley River within Charleston Harbor (Charleston, S.C., USA). Screening was performed in sediment fuel cells (FIG. 2). In particular, sediment fuel cells similar to those described by Holmes et al. (2004) were constructed as follows: sediment free of shells and plant detritus was made homogenous by stirring and was added to the 250 ml mark of 600 ml beakers, which were then filled to the 500 ml mark with harbor water. Approximately 50 ml of ddH₂O was added daily to replace water lost to evaporation. Placing a flask of water in the oven helped to minimize evaporation in the sediment fuel cells. Graphite electrodes with a surface area of 6.7 cm² were prepared with marine-grade wire as previously described (Milliken, 2007). Those serving as anodes were placed 5 cm below the surface of the sediment, 4 cm away from the sides of the beakers and 2 cm away from the bottom of the beakers. Cathodes of the same size were suspended in the overlying water 2 cm above the sediment surface and 7 cm from the buried anodes. The electrodes were connected through a 1000-Ohm resistor, which was maintained at the temperature applied to the fuel cells (FIG. 4). To determine the effect of temperature on the load, a 1000-Ohm resistor was incubated at 60° C., which resulted in a 0.5% decrease in resistance versus when the resistor was maintained at 22° C. The sediment fuel cells were incubated in an incubator-oven that was pre-set at the designated thermophilic temperature. Air was delivered to the overlying water continuously at 140 ml/min though surgical tubing by an aquarium pump. A set of three killed-cell control sediment fuel cells were prepared similarly but were treated with 1% formaldehyde prior to study.

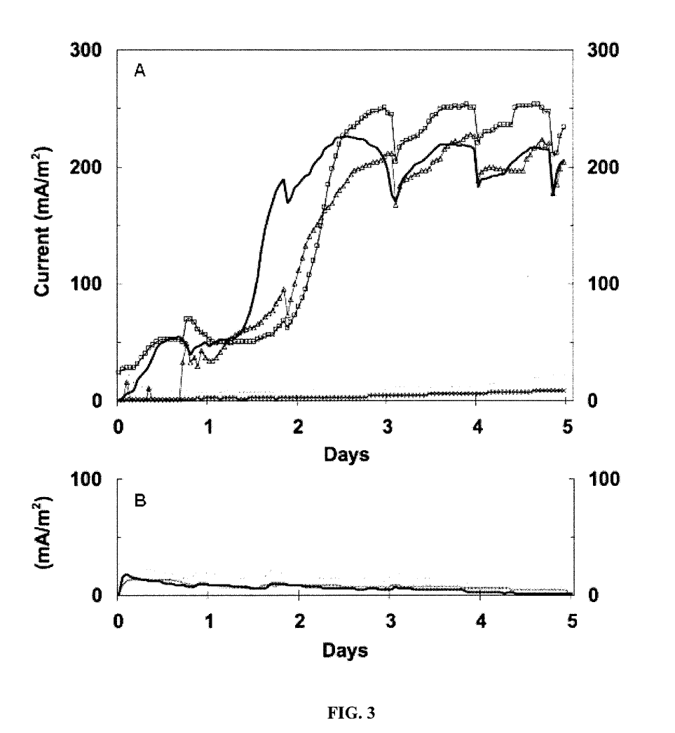

The sediment supplied carbon and energy to the electricigenic bacteria while the anode in the anoxic sediment served as a terminal electron acceptor. The cathode in the overlying water was bathed in oxygenated water, which presented the system with a large difference in oxidation/reduction potential between the sediment and water environments. In this way, the anaerobic electricigens indirectly used oxygen as an electron acceptor. The electricigens were then enriched on the surface of the anode. FIG. 3 shows the electricity generated with sediment used to enrich for microbial communities of Thermincola spp. and Deferribacteres. The figure demonstrates that the electric currents established with sediment fuel cells incubated at 60° C. far exceeded that produced by cells incubated at 22° C. Comparison of several sediment fuel cells incubated at a range of temperatures showed 60° C. to be near optimal for electricity generation (FIG. 4).

b. Single-Chamber Fuel Cells

After a stable current has developed, the anodes can be transferred away from the sediment and into a single chamber fuel cell for further enrichment of the electricigenic bacteria. Single-chamber fuel cells (25 ml total volume) made of glass were prepared as described previously (Milliken, 2007). The anodes were identical to those used in the sediment fuel cells and the cathodes were made of platinum-carbon cloth with 0.5 mg Pt/cm² using 10% Pt on Vulcan XC-72 (E-Tek, Somerset, N.J., USA) and had a surface area of 1.7 cm². Nafion®117 (The Fuel Cell Store, Boulder, Colo., USA) was clamped to the inner surface of the cathode. A minimal anaerobic medium (ECl, pH 6.8 (Berkaw et al, 1996)) without any soluble synthetic mediators, resazurin, sulfide or cysteine was prepared under strict anoxic conditions under N₂:CO₂ (80:20). Before transfer of the sediment fuel cell anodes, the single-chamber assembly was wrapped in foil and autoclaved for 45 min and then placed for at least 12 hours in an anaerobic Coy chamber (Grass Lake, Mich., USA). The anodes were taken directly out of the sediment, gently shaken to remove excess sediment, and placed into the single-chamber fuel cell under positive pressure N₂:CO₂ (80:20) supplied by canula. The system was filled with 20 ml of medium, sealed with a black butyl stopper, and placed in a 60° C. incubator. Medium within the anode chamber was exchanged every 2 to 3 days by syringe under an atmosphere of N₂:CO₂ (80:20). At the time of exchange, the spent medium had a pH of 6.3 to 6.5 and had lost 8 to 10 ml of volume. Replacement of the medium restored the pH to 6.8 and the volume to 20 ml. To prepare a sterile, killed-cell control, an anode from an electricity-producing sediment fuel cell was sealed in an anaerobe tube with 10 ml of medium and autoclaved for 45 min. This anode was then transferred to a fuel cell assembly as described for the live systems.

c. Monitoring Electricity

Voltage measurements on sediment fuel cells and single chamber cells were made as described previously (Milliken, 2007). Continuous 60-minute interval voltage measurements across a 1000-Ohm load resistor were taken throughout the experiments. Current (I) was calculated as I(mA)=V(mV)/R(Ohms) where V is the voltage and R is the external resistance. Power (P) in milliwatts (mW) was calculated as P(mW)=I² (mA)R(Ohms). Current and power densities were normalized to the surface area of the electrodes.

d. Monitoring Acetate and Electron Recovery

Acetate measurements were made by application of fuel-cell medium to an ion chromatograph using methods previously described (Milliken, 2007). An eight-electron oxidation of the acetate to CO₂ was used in the calculations. The electron recovery (Coulombic efficiency, Ec) was based on changes in acetate consumption and current across 1000 Ohms over time, where Ec=Coulombs of current divided by Coulombs available based on measured acetate consumption. Conversions to Coulombs were based on 1 C=1 A×1 s, 1 C=6.24×10¹⁸ electrons, 1 mol=6.02×10²³ electrons and therefore 96,500 C/mol. Methane analysis was done by application of 50 μl of headspace gases from the fuel cells to a gas chromatograph (Hewlett-Packard 6890) equipped with a flame ionization detector (Cutter et al., 2001).

e. Scanning Electron Microscopy

An anode from an acetate-fed cell, following 10 exchanges with sediment-free media, was immersed in 2% glutaraldehyde in sodium cacodylate buffer overnight, then chemically dehydrated with hexamethyldisilazane overnight. An SC7640 desktop sputter coater (Polaron, Hertfordshire, UK) was used to coat the samples with approximately 100 Angstroms of gold and palladium mix. The sample was then analyzed in a JEM-5410LV Scanning Electron Microscope (JEOL, Inc., Tokyo, JAPAN) at 15 kV accelerating voltage.

f. Sequencing and Analysis of the 16S rRNA Genes

Amplified ribosomal DNA restriction analysis (ARDRA). The anode of an electricity-generating single-chamber fuel cell, fueled with acetate (25 mM) and receiving 10 exchanges of sediment-free media, was aseptically scraped with a sterile scalpel to collect the community that had formed a biofilm on the electrode. Whole genomic DNA extraction from the microbial community was performed according to the manufacturer's instructions with a PowerSoil DNA Isolation kit (Mo Bio Laboratories, Inc., Carlsbad, Calif., USA). PCR amplification of the 16S rRNA gene used the universal primers 27F (5′-AGAGTTTGATCMTGGCTCAG-3′; SEQ ID NO: X) and 1492R (5′-GGYTACCTTGTTACGACTT-3′; SEQ ID NO: Y) and the Choice Taq Blue Mastermix (Denville Scientific, Inc., Metuchen, N.J., USA). The PCR method performed on a GeneAmp PCR system 9700 (Applied Biosystems, Foster City, Calif., USA) had an initial denaturation step of 1:30 at 94° C., 30 cycles of 94° C. for 30 s, 55° C. for 30 s, and 72° C. for 30 s, followed by the final extension step of 72° C. for 7 min. The PCR product was ligated and cloned using the pGEM-T Easy Vector System II according to the manufacturer's protocol (Promega, Madison, Wis., USA.). Positive clones were screened on LB/ampicillin/IPTG/X-gal (LAIX) plates and 80 clones were grown overnight in an LB/amp 100 media. A culture PCR was performed to amplify the 16S rRNA gene needed for restriction analysis and sequencing. The same PCR conditions were used as described above with the slight modification of the PCR method as follows: 95° C. for 3 min followed by 40 cycles of 95° C. for 30 s, 55° C. for 30 s, and 72° C. for 1 min, and the final extension step of 72° C. for 5 min. The PCR product in this step was subjected to two separate restriction digests of the HhaI and HaeIII restriction enzymes. The restriction digest was performed at 37° C. for 2 hrs in 1.5 μl of supplied Buffer C, 1.5 μl 10×BSA, 0.1 μl restriction enzyme, and 11.9 μl PCR product. Each restriction digest was visualized on a 2% Trevigel (Trevigen, Inc., Gaithersburg, Md., USA) in 1×TAE buffer and the isolates with distinct patterns in each digest were selected for sequencing.

One to 4 clones of each of the 11 different representative RFLP patterns were selected for sequence analysis. Plasmid DNA was isolated using the Qiaprep Spin Miniprep kit (Qiagen, Inc., Valencia, Calif., USA) and sent to the BioAnalytical Services Laboratory at the University of Maryland Biotechnology Institute. The samples were sequenced on an ABI 3130 XL Genetic Analyzer using the sequencing primers M13F and M13R.

The consensus sequences for each of the 11 different RFLP patterns were assembled using the SeqMan program in the DNASTAR software package (DNASTAR, Inc. Madison, Wis., USA.). Each consensus sequence contained at least 1460 base pairs and was subjected to BLAST and RDP analysis. Phylogeny was determined with the Ribosomal Database Projects' Classifier (Wang et al., 2007) and Seqmatch (Cole et al., 2007). The 16S rRNA gene sequences were compared to the GenBank database and similarity scores were calculated using BLAST analysis (Atschul et al., 1990). The DNASTAR software package previously mentioned was used for alignment of the 16S rRNA genes using the MegAlign program and the CLUSTALW algorithm. The nucleotide sequences generated in this study were submitted to GenBank under the accession numbers EU194827 through EU194837.

2. Results

a. Electricity Generation Under Thermophilic Conditions

Sediment fuel cells, constructed with marine sediment and operated at 60° C. without added energy sources or synthetic electron-carrying mediators, generated direct electric current well above that produced by counterparts incubated at 22° C. (FIGS. 3A-B). Maximum currents per m² of anode surface were established between 2 and 5 days and ranged from 209 to 254 mA/m² (29 to 43 mW/m²) for the triplicate live cells incubated at 60° C. Background currents for the killed-cell control MFCs leveled off between 3 and 8 mA/m². Similarly prepared sediment fuel cells (again in triplicate) incubated at 22° C. generated 10 to 22 mA/m² within 5 days (FIG. 3A), an order of magnitude less than produced by the thermophilic cells. Electricity generation peaked at 60° C. in relation to other temperatures (FIG. 4), but was sustained at 75° C. Comparison of several sediment fuel cells incubated at a range of temperatures showed 60° C. to be near optimal for electricity generation (FIG. 4). Current ceased when active cells were exposed to 90° C. Summation of the data from FIGS. 3A-B and FIG. 4 show that when all parameters but temperature were held constant the thermophilic sediment fuel cells generated nearly 10-fold higher current than the mesophilic counterparts.

b. Electricity Generation without Sediment in Single-Chamber Cells

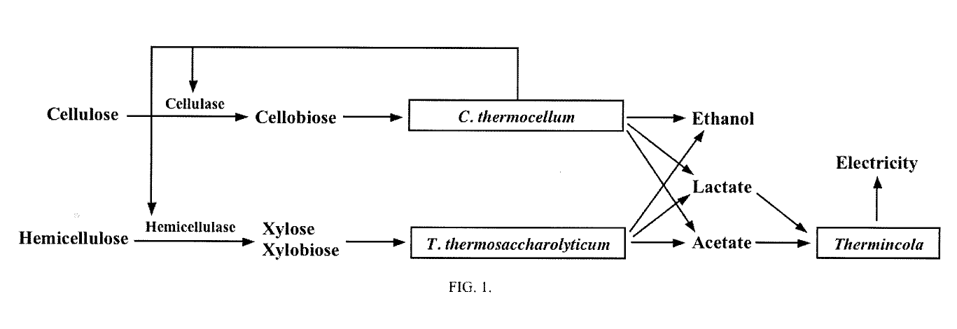

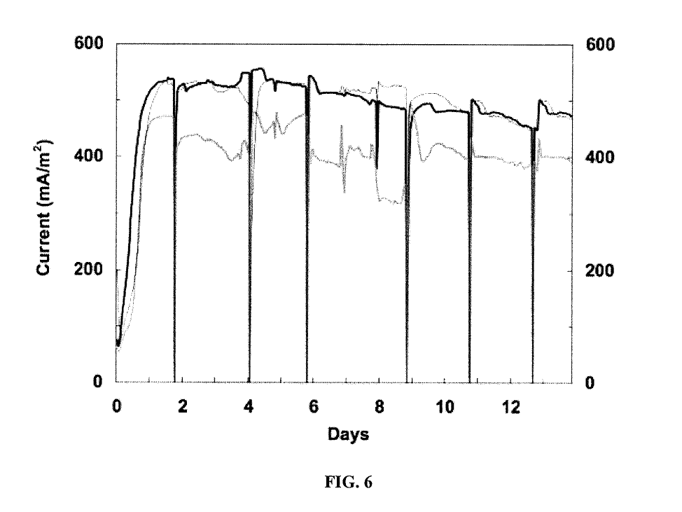

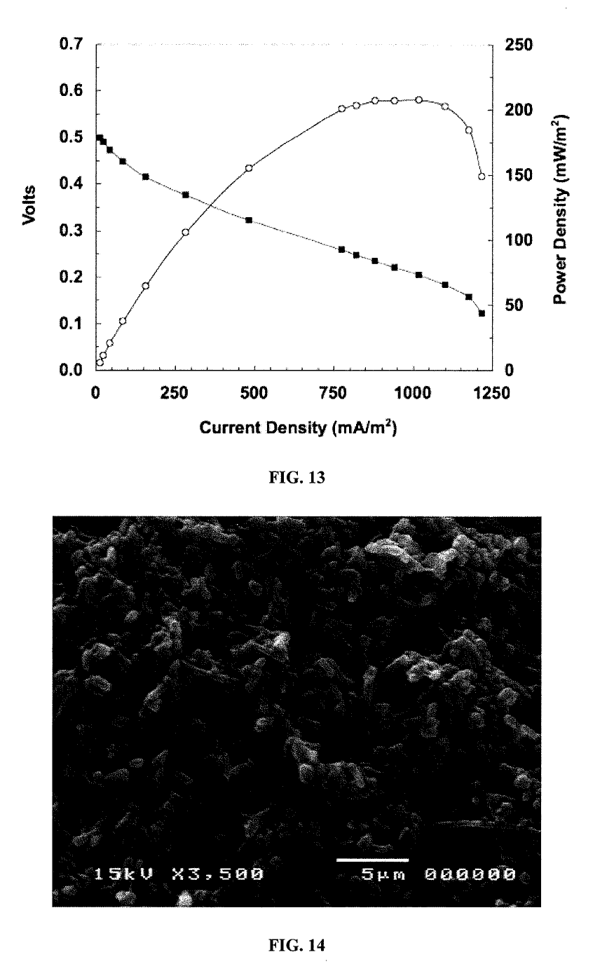

Anodes from the sediment fuel cells described in FIG. 1 were transferred into single-chamber fuel cells equipped with air-bathed, Pt-carbon cloth cathodes (FIG. 5). This increased the availability of oxygen to the cathode and enabled the examination of the thermophilic microbial electrode reduction in the absence of sediment and externally-supplied mediators. The anodes were gently shaken in order to minimize transfer of sediment to the single chamber cells, and anaerobic minimal medium plus 25 mM sodium acetate was added to each of the cells, which were then incubated at 60° C. In less than two days, the current produced by these cells had stabilized at 478 to 537 mA/m² of anode surface (FIG. 6). Current generated at 60° C. by single chamber cells with anodes 1^{st} established in sediment fuel cells. The fuel used without sediment in the single chamber cells was 25 mM acetate, which was supplied with each exchange of the medium (vertical lines). FIG. 6 shows data from an anode that was transferred from a thermophilic sediment fuel cell to a single chamber cell fed acetate. Sediment transfer to the cell was minimized and residual sediment was removed with each replacement of spent media (the vertical lines in the figure). The electricity generation with anodes started in marine sediment fuel cells was sustainable without adding mediators to single-chamber fuel cells fueled with acetate (FIG. 6), indicating that thermophilic electricigens are present that do not require an exogenous mediator. A polarization and power curve analysis normalized to the surface area of the anode (FIG. 13) revealed an open-circuit voltage of approximately 0.5 Volts and a maximum power density of 207 mW/m² of anode surface. The surface area of the cloth cathode was approximately 4-fold less than that of the anode; therefore the power density per cathode surface area was 815 mW/m².

c. Acetate as a Fuel

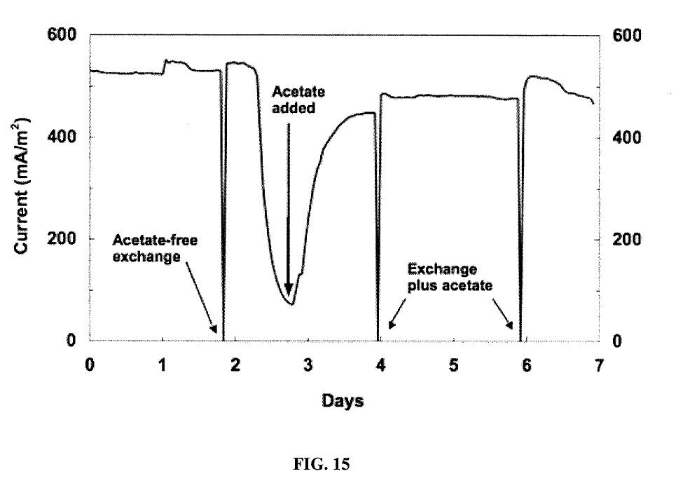

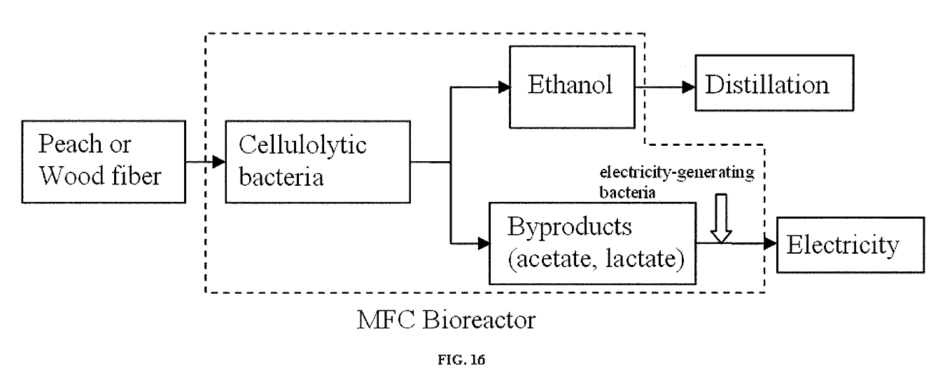

Current was immediately restored in acetate-fed, single-chamber fuel cells following successive exchanges of the medium and this resulted in the elimination of visible sediment (FIG. 6). This also resulted in a very heavy biofilm of rod-shaped bacteria on the surface of the anode (FIG. 14). A 10-15% decline in current was observed over a two-week period, but the current could be restored if the Nafion membrane was replaced after two weeks. This bacterial community could then be transferred from cell to cell in ECl medium or to a serum bottle containing ECl medium with 15 mM sodium acetate and 10 mM sodium fumarate and then back to a fuel cell and electricity was again generated. The community has been thus transferred and maintained without sediment for 1 year and more than 10 transfers and has continued to produce electricity as demonstrated in FIG. 6. In one of the single-chamber fuel cells the microbial community was starved for fuel. The addition of sodium acetate after the current had declined caused a rapid restoration of electricity generation, indicating that acetate was serving as the fuel for electricity production by the thermophilic bacterial community (FIG. 15). FIG. 6 shows data from an anode that was transferred from a thermophilic sediment fuel cell to a single chamber cell fed acetate. Sediment transfer to the cell was minimized and residual sediment was removed with each replacement of spent media (the vertical lines in the figure). The Coulombic efficiency (electron recovery) from acetate was 35.5±9.6% (n=6) and was determined from several different MFCs by measuring the acetate consumption over time while current remained above 450 mA/m². Methane was not detected in the headspace of the cells (detection limit of 0.5 μmoles). The most likely explanation for the low recovery of electrons is that the mixed microbial community includes aerobic or microaerophilic acetate-consuming bacteria and that oxygen enters during the manipulation (medium exchange) and operation of the cell.

The visible biofilm developed on the surface of the anode could be scraped and used to inoculate a sterile anode in a new single chamber fuel cell. This procedure was followed by transfer of the biofilm to media containing acetate plus 100 mM-amorphous FeIII oxide. This and all subsequent culture work was done at 60° C. After several days the precipitated iron turned black. A 10% transfer of the culture was then made to media containing acetate plus 10 mM sodium fumarate and after a few days this culture became turbid. The culture was diluted to extinction in acetate plus fumarate media. Growth at the 10-8 dilution was transferred into a new single chamber MFC and electricity was established within a day. The entire procedure (growth in liquid media, dilution, growth on electrode) was repeated twice. The culture is presently being grown on agar media containing acetate plus fumarate. The combination of growth conditions including thermophilic with an electrode as the sole electron acceptor is highly selective.

d. Community Analysis of an Acetate-Fueled Cell

Cloning of an acetate-fed community from the surface of a graphite anode resulted in 80 clones with 1460 bp of 16S rRNA gene sequence (Table 2, supra). All possessed sequences with a clear majority of Firmicutes (64 clones). Of the Firmicutes, 48 had identical RFLP patterns (B) and sequence most similar (99%) to that of Thermincola carboxydophila strain 2204. The 16S rRNA genes from 6 other clones produced a different RFLP pattern (E), yet the sequence was also 99% similar to that of T. carboxydophila strain 2204. Five more clones (RFLP patterns F and H) held sequence most similar to T. carboxydophila, but more distantly (88 to 90% similarity). The remainder of the Firmicutes (RFLPs G, I and J) was most related to a series of uncultured bacteria. All of the remaining 12 clones (RFLPs A, C, D and K) held 16S rRNA gene sequences most related to uncultured Deferribacteres (87 to 96% similarity).

The examination of the 16S rRNA genes from an acetate-consuming community on the anode of a fuel cell revealed a community dominated by Gram positive bacteria. Most of the clones (61 of 80) held DNA most similar to that of Thermincola carboxydophila (99% similarity). Two Thermincola spp., T. carboxydophila and T. ferriacetica, are described in the literature (Sokolova et al., 2005; Zavarzina et al., 2007). Both are Gram positive spore-forming moderate thermophiles that have been isolated from terrestrial hot springs. Three more clones (RFLP G) are also most related to bacteria from a thermophilic environment, in this case an uncultured Firmicute from a terephthalate-degrading thermophilic community grown in an anaerobic reactor (Chen et al., 2004). The five remaining Firmicute-related clones could not be identified with thermophiles or mesophiles based on their most related sequences in Genbank. Twelve clones (RFLPs A, C, D and K) did not contain DNA of Gram positive bacteria. Instead, these were most related to uncultured Deferribacteres (87 to 96% similarity). Deferribacter spp. are Gram negative moderate thermophiles isolated from deep subsurface waters and other thermal environments (Greene et al., 1997; Miroshnichenko et al., 2003; Takai et al., 2003). Six of the clones (RFLPs C and D) were most closely related to two uncultured bacteria discovered in a thermophilic MFC inoculated with brewery waste (Jong et al., 2006).

It is apparent that the community described consists of generally two types of bacteria: Gram positive bacteria most related to Thermincola spp., which were dominant in the clonal analysis, and Gram negative bacteria related to Deferribacter spp. All cultured strains of these genera are known to be thermophilic. Marine sediments have been used to enrich electricity-generating communities under mesophilic conditions (Bond et al., 2002; Holmes et al., 2004) but Thermincola and Deferribacter are not part of these communities. Deferribacter thermophilus (Greene et al., 1997), D. abyssi (Miroshnichenko et al., 2003), and Thermincola ferriacetica (Zavarzina et al., 2007) are capable of using acetate as a carbon and energy source and insoluble iron external to the cell as an electron acceptor. Although it is not always the case, reduction of iron external to a bacterial cell is a common property of electricigenic bacteria (Lovley, 2006; Yan et al., 2007).

e. Electricity Generation with Cellulose

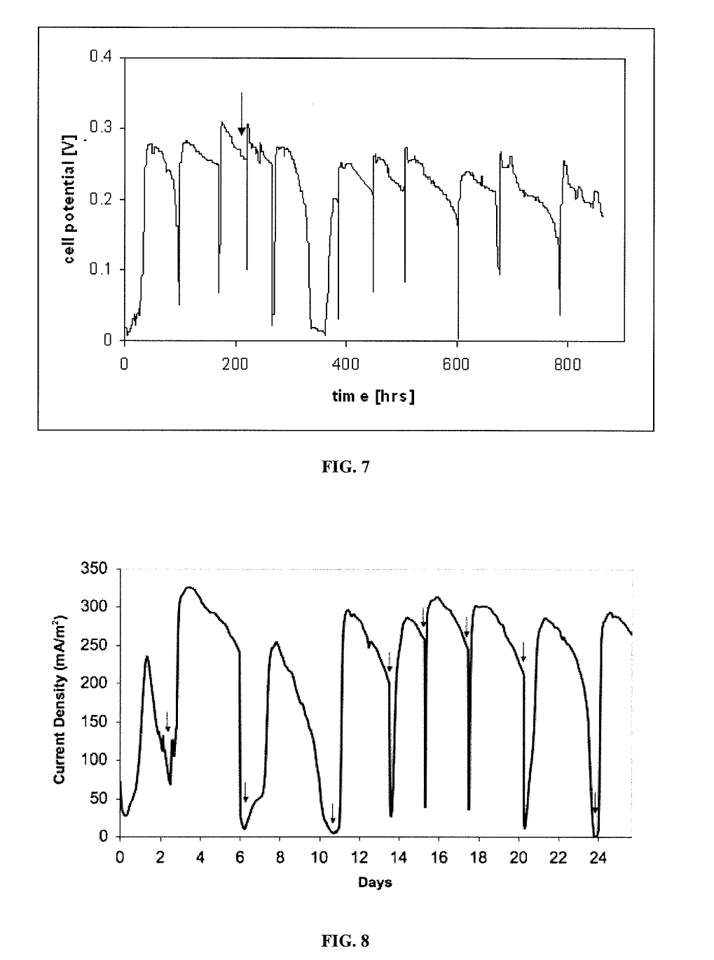

Cellulose can also serve as a fuel source for a thermophilic electrode-reducing community. As FIG. 7 shows, current could be sustained with cellulose added as a sole carbon and energy source to a single-chamber cell. In this case, the biocatalyst was first enriched in a sediment fuel cell, transferred to a single-chamber cell and fueled with acetate, the community was then maintained in medium with acetate plus sodium fumarate or insoluble Fe(III). This community was then autoclaved to recover autoclave resistant spores, grown on acetate plus fumarate containing agar plates, transferred to acetate plus fumarate and acetate plus insoluble (Fe(III) medium and after growth autoclaved again. The resulting culture was then maintained in acetate plus insoluble Fe(III) medium before testing in the fuel cells. The culture was not exposed to cellulose until the test recorded in FIG. 7. The identity of the culture is being determined now, but before autoclaving the culture consisted of Thermincola spp. and Deferribacteres. Of these, only Thermincola spp. are known to possess autoclave-resistant spores. Until now, no cellulolytic Thermincola sp. had been reported. The culture was started in the MFC with only acetate as fuel, but this medium was washed away and replaced with cellulose-containing medium (FIG. 7). From that point forward the culture continued to produce electricity from cellulose. No ethanol was produced.

f. Community Analysis of an Cellulose-Fueled Cell

The community of Thermincola spp. enriched from marine sediment by the inventors was initially enriched on an electrode of a MFC supplied with acetate as fuel. The culture was further enriched by 1) repeated serial transfer from MFC to culture media with acetate and insoluble iron, 2) autoclaving for 30 minutes, 3) cultivation in a MFC with acetate as fuel, 4) isolation as a colony on agar containing media with acetate and fumarate, 5) autoclaving again, 6) cultivation in a MFC and again in acetate plus insoluble iron media. After this enrichment/isolation procedure, the culture would use acetate or cellulose as fuel and generate electricity in a MFC. The culture contains only Gram positive rods that produce autoclave resistant spores. Before autoclaving, the community consisted of Thermincola spp. and Deferribacteres (Table 2). After autoclaving, only Thermincola spp. and other Firmicutes remained. The 16S RNA sequences of the Thermincola spp. and other Firmicutes are found under GenBank Accession Nos. EU194830, EU194831, EU194832, EU194833, EU194835, EU194836, EU194837.

The inventors have recently documented operation of sediment MFCs at thermophilic temperatures and have identified the first thermophilic electricigen, Thermincola ferriacetica (DSMZ 14005). This organism is unique as an electricigen in that it is a thermophile, is Gram positive, and produces autoclave resistant spores. It does not require the addition of a soluble mediator to transfer the electrons to the electrode. They have also demonstrated that a group of Thermincola spp. (16S RNA sequences listed under GenBank Accession Nos. EU194830, EU194831, EU194832, EU194833) most related to Thermincola ferriacetica (88 to 99% similarity by 16S rRNA gene sequence) will generate electricity with acetate or cellulose as a fuel. These bacteria are also Gram positive thermophiles that produce autoclave resistant spores and do not require the addition of a soluble mediator to transfer electrons to an electrode.

g. Electricity Generation by Thermincola ferriacetica

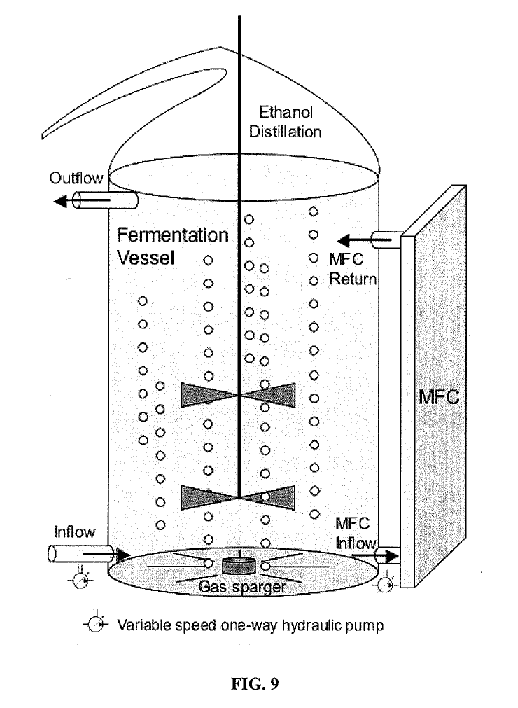

The inventors have demonstrated for the first time, electricity generation by a pure culture of a thermophile. Thermincola ferriacetica strain Z-0001 (DSMZ 14005) is a strict anaerobe and can use Fe(III), Mn(IV) or anthraquinone-2,6-disulfonate (AQDS) as electron acceptors and acetate as an electron donor (Zavarzina et al., 2007). The direct transfer of T. ferriacetica (DSMZ 14004) grown with insoluble iron oxides to a fuel cell has resulted in the generation of electricity (FIG. 8). Iron could be mediating electron transfer to the electrode. This may be a primary method of electron transfer by such Gram positive bacteria in a sediment fuel cell and may explain why Thermincola is so prevalent in 16S rRNA gene analysis in Table 2. The inability of T. ferriacetica to grow with an electron acceptor other than Fe(III), Mn(IV) or AQDS complicates the investigation of this organism as an electrode reducing bacterium. At this stage it appears that mediation of electron flow through iron external to the cell may be a method of electrode reduction. This apparently would still require electron transfer to the surface of the Gram positive wall, and how this would occur is not clear. Furthermore, this means that the bacterium would be operating with an exogenous mediator, albeit a very prevalent, inexpensive and natural one.

However, mediation by iron of electrons from T. ferriacetica to the electrode may be too simplistic of an explanation. Extensive washing of the anode with media removed any visible colloidal iron while currents remained steady (FIG. 8). In addition, following more than 10 exchanges of the media, the spent media from an active MFC with T. ferriacetica was transferred to a second fuel cell and electricity was generated. Although iron would still be present, these results suggest that it is possible that a second mechanism, one not so reliant on iron, may be at work. Perhaps T. ferriacetica is generating a soluble mediator similar to what has been shown with a Pseudomonas sp. (Rabaey et al., 2005). Further examination of the bacterium now being isolated (see electricity generation with cellulose above), which appears to be a Thermincola and can grow without iron as an electron acceptor, will hopefully permit an understanding of how this Gram-positive organism mediates electron flow to an electrode.

An examination of marine sediment from temperate waters (Charleston, S.C., USA) proved to be a good source of thermophilic electrode-reducing bacteria. Electric current normalized to the surface area of graphite electrodes was approximately ten-times greater when sediment fuel cells were incubated at 60° C. (209 to 254 mA/m²) versus 22° C. (10 to 22 mA/m²). Electricity-generating communities were selected in sediment fuel cells and then maintained without sediment or synthetic electron-carrying mediators in single-chambered fuel cells. Current was generated when cellulose or acetate was added as a substrate to the cells. The 16S rRNA genes from the heavy biofilms that formed on the graphite anodes of acetate-fed fuel cells were cloned and sequenced. The preponderance of the clones (54 of 80) was most related to a Gram-positive thermophile, Thermincola carboxydophila (99% similarity). The remainder of clones from the community was most related to T. carboxydophila, or uncultured Firmicutes and Deferribacteres. Overall the data indicate that temperate aquatic sediments are a good source of thermophilic electrode-reducing bacteria.

The single chamber fuel cells described above for the isolation and testing of Thermincola ferriacetica and other thermophilic electricigens can be used to select for microbial communities that more effectively convert cellulose to ethanol and electricity. In this case mixed inocula from various sources (soils, sediments, etc.) would be added to fuel cells that are inoculated with C. thermocellum and T. thermosaccharolyticum and incubated at 60° C. The MFCs would then be operated in semi-batch mode through the periodic replacement of the medium and if need be re-inoculation of the fermentative bacteria. The population of thermophilic electricigens would then develop in response to the fermentative bacteria. The other driver is the pretreated biomass slurry. This could be used in combination with the fermentative bacteria or alone in order to select for competent thermophilic electricigens.

### Example 2

### Simultaneous Production of Ethanol and Electricity in Microbial Fuel Cells with Cellulose as a Carbon Source Under Thermophilic Conditions

1. Methods

a. Microorganisms

Electricigenic microorganisms used in this example were either a pure culture of Thermincola ferriacetica (DSM 14005) which was purchased from the German Resource Centre for Biological Material or a mixed culture enriched and isolated from marine sediment as shown in EXAMPLE 1. The both cultures were grown in a serum bottle containing 10 mM of sodium acetate, 15 mM of insoluble iron oxyhydroxide, 0.1% of yeast extract with the ECL medium at 60° C.

Ethanologenic microorganisms used in this example was a pure culture of Clostridium thermocellum 651 (ATCC 27405) which was purchased from the American Type Culture Collection. The inventors also attempted to use Thermoanaerobacterium thermosaccharolyticum NCA 3814 (ATCC 7956) in combination with C. thermocellum. T. thermosaccharolyticum is known to convert pentose which is produced in the metabolic process of cellulose by C. thermocellum into ethanol. However, due to its slow reaction, the inventors did not observe a positive effect of T. thermosaccharolyticum addition, and therefore, they do not show the result for this particular case.

b. Single-Chamber Fuel Cells

Single-chamber fuel cells as described in EXAMPLE 1 were used in this example. One negative aspect of using this air-cathode type fuel cell is permeation of ethanol through the Nafion® membrane. To capture all ethanol produced in the microbial fuel cell, including this permeated portion of ethanol, the fuel cell was placed in a polyethylene plastic bag containing 10 ml of water and sealed by a laminator in some cases. A 1000 Ohm resistor was typically used except for one case where 10000 Ohm resistor was used. All of the fuel cells were incubated at 60° C.

Each microbial fuel cell was initially inoculated with the culture of electricigenic microorganisms only. After a few medium exchanges with addition of 10 mM acetate, biofilm on the anode was assumed to be established. At this point, when the medium was exchanged, 0.1 g/20 ml of cellulose powder was added instead of sodium acetate. At the same time, a culture of C. thermocellum was added at 5-10 vol %. While monitoring simultaneous production of ethanol and electricity from these microbial fuel cells, 10% of spent medium was mixed left and mixed with 90% of new medium, and 0.1 g/20 ml of cellulose was added without re-inoculation of C. thermocellum.

c. Voltage Measurements, Quantification of Ethanol and Acetate

Voltage measurements on these microbial fuel cells were automatically conducted every 30 min using a multimeter (Keithley 2700) connected to a computer. For the fuel cells sealed in a plastic bag, voltage measurements were conducted manually when the fuel cells were taken out of the bag for sampling.

Concentrations of ethanol and acetate produced in the microbial fuel cells were determined by a gas chromatograph (Hewlett Packard 5890 Series II) with a flame ionization detector and a HP-FFAP column (Hewlett Packard 19095F-123, 30 m×530 μm×1 μm). Liquid samples (1 ml each) were aseptically taken from the microbial fuel cells and filtered using a 0.2 μm syringe filter. When the fuel cell was placed in the plastic bag, water in the bag was also sampled and filtered. The filtered samples were acidified using 10 wt % formic acid by mixing them at 9:1 volumetric ratio. The acidified sample (1 μl) was injected into the gas chromatograph using an autosampler (Hewlett Packard GC system injector 18593B). The open temperature program was as following: 80° C. for 1 min, 20° C./min to 120° C., 6.13° C./min to 205° C., 205° C. for 2 min. Helium was used as a carrier gas at a constant pressure of 4.1 psi. Air and hydrogen gases were used for the detector, and nitrogen was used as a make-up gas. The injection was split at a ratio of 9:3:1. The detector temperature was 230° C. The ethanol and acetate calibration curves were linear in the concentration ranges of interest.

2. Results

a. Electricity Generation from the Microbial Fuel Cells

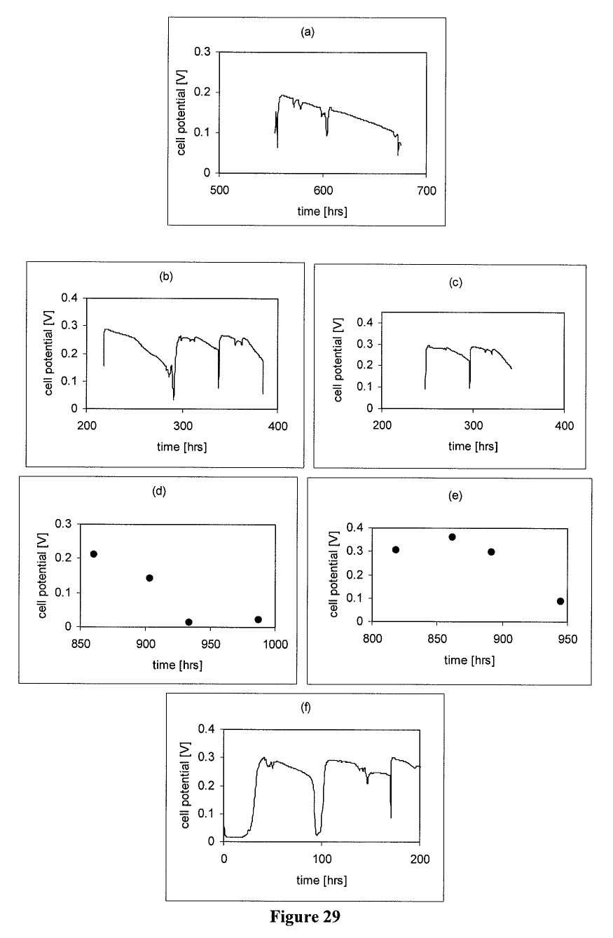

FIG. 29 shows electricity generation from these microbial fuel cells. In general, the cell potential of these microbial fuel cells was not negatively affected by the inoculation of C. thermocellum. One control fuel cell containing only the mixed culture of electricigenic microorganisms with cellulose as a carbon source revealed that the mixed culture can consume cellulose and produce acetate. Therefore, the mixed culture of electricigens does not necessarily depend on C. thermocellum because the mixed culture can use either cellulose, acetate produced by itself, or acetate produced via cellulose fermentation by C. thermocellum. In the case of T. ferriacetica, however, it is known that it cannot consume cellulose. Therefore, T. ferriacetica was assumed to live on acetate which was produced via cellulose fermentation by C. thermocellum.

b. Production of Ethanol from the Microbial Fuel Cells.

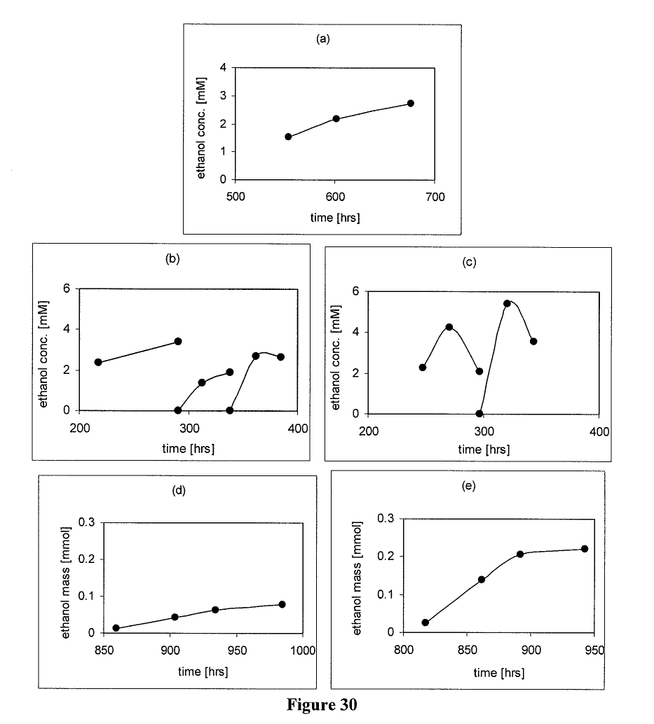

FIG. 30 shows ethanol production from these microbial fuel cells. The result supports that C. thermocellum can survive and produce ethanol in the microbial fuel cell's environment. Even after medium exchanges, these microbial fuel cells which were once inoculated with C. thermocellum produced ethanol repeatedly. The apparent decrease of ethanol concentrations in some cases are due to permeation of ethanol through the membrane out of the fuel cell. In the experiment where the fuel cells were sealed in a plastic bag, total mass of ethanol produced kept increasing, but the production rate decreased toward the end. This decrease of ethanol production is likely due to a decrease of pH in the medium. When a 10000 Ohm resistor was used instead of 1000 Ohm, ethanol production was higher. When a pure culture of T. ferriacetica was used, ethanol production was smaller than when a mixed culture of electricigens was used. An increase of acetate concentrations in these microbial fuel cells was also observed, suggesting that consumption of acetate by the electricigens was slower than its production by C. thermocellum.

### Example 3

### Simultaneous Production of Ethanol and Electricity in Microbial Fuel Cells with Crushed Peach as a Carbon Source Under Thermophilic Conditions

1. Methods

a. Microorganisms

Microorganisms used in this example are basically the same as those in EXAMPLE 2. A mixed culture enriched and isolated from marine sediment was used as electricigens. A pure culture of Clostridium thermocellum 651 was used as ethanologens. However, the inventors never used a pure culture of Thermincola ferriacetica in this example.

b. Medium Preparation

To prepare the medium containing peach, frozen peach without skin and seeds was thawed, and 10 g of wet peach was crushed and mixed with 90 g of ECL medium using a blender. This peach-containing medium was autoclaved at ˜110° C. for 15 min. According to Nutrition Facts provided by the peach supplier, 100 ml of this medium should contain 0.93 g of total carbohydrate, 0.14 g of fiber (cellulose), and 0.64 g of total sugars. C. thermocellum was expected to consume both sugars and cellulose in this medium, producing ethanol and acetate. The mixed culture of electricigens was expected to consume sugars and acetate produced by C. thermocellum to produce electricity and carbon dioxide

c. Single-Chamber Fuel Cells

The same kind of single-chamber fuel cells as described in EXAMPLE 2 were used in this example. No attempt was made to capture a portion of ethanol permeating through the Nafion® membrane. A 1000 Ohm resistor was used, and all fuel cells were incubated at 60° C.

After a biofilm of the electricigenic microorganisms on the fuel cell's anode was established as described in EXAMPLE 2, the spent medium was completely removed, and the fuel cell was rinsed with 10 ml of ECL medium twice. This was to remove all residual carbon sources, whether acetate or cellulose, and make sure that the peach would be the only carbon source in the system. After the rinsing, ECL medium containing 10 wt % of peach was added to the fuel cell. At the same time, a culture of C. thermocellum was added at 5 vol % to the fuel cell.

d. Voltage Measurements, Quantification of Ethanol and Acetate

Voltage measurements and determination of ethanol and acetate in the microbial fuel cell was performed in exactly the same manner as in EXAMPLE 2.

2. Results

a. Electricity Generation from the Microbial Fuel Cells

FIG. 27(i) shows electricity generation from this microbial fuel cell. It was confirmed that the mixed culture of electricigens can generate electricity using peach as a sole carbon source in this microbial fuel cell. Unlike cellulose, addition of peach had a negative effect on the electricity generation from this microbial fuel cell, decreasing the cell potential by 50-70% relative to that of acetate-fed microbial fuel cells without ethanologens. This negative effect is believed to due to other components of peach such as pectin which the electricigens cannot consume, and removal of such components in a pretreatment process should improve the performance of this microbial fuel cell.

b. Production of Ethanol from the Microbial Fuel Cells.

FIG. 27(ii) shows ethanol production from this microbial fuel cell. It is clear that C. thermocellum can survive and produce ethanol in the microbial fuel cell's environment. Compared with the cellulose-fed microbial fuel cells in EXAMPLE 2, peach seems to be as good carbon source as cellulose for ethanol fermentation by C. thermocellum.

All of the methods and apparatus disclosed and claimed herein can be made and executed without undue experimentation in light of the present disclosure. While the compositions and methods of this invention have been described in terms of particular embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### H. References

The following references, to the extent that they provide exemplary procedural or other details supplementary to those set forth herein, are specifically incorporated herein by reference.
U.S. Pat. No. 3,990,944
U.S. Pat. No. 3,990,945
U.S. Pat. No. 5,000,000
U.S. Pat. No. 5,028,539
U.S. Pat. No. 5,424,202
U.S. Pat. No. 5,482,846
U.S. Pat. No. 7,271,244
U.S. Patent Publn. 20020137154
U.S. Patent Publn. 20030054500
U.S. Patent Publn. 2006-0011491
U.S. patent Ser. No. 11/180,454
Aden et al., U.S. DOE National Renewable Energy Laboratory, 2002.
Angenent et al., Trends in Biotechnology, 22:477-485, 2004.
Atschul et al., J. Mol. Biol., 215:403-410, 1990.
Berkaw et al., Appl. Environ. Microbiol., 62(7):2534-2539, 1996.
Bond et al., Appl. Environ. Microbiol., 69:1548-55, 2003.
Bond et al., Appl. Environ. Microbiol., 71:2186-9, 2005.
Bond et al., Science, 295:483-485, 2002.
Chen et al., Microbiology, 150:3429-3440, 2004.
Cheng et al., Environ. Sci. Technol., 40:364-369, 2006.
Choi et al., Bull, Korean Chem. Soc., 25:813-818, 2004.
Cole et al., Nucleic Acids Res., 35:D169-D172, 2007.
Cutter et al., Environ. Microbiol., 3(11):699-709, 2001.
Demain et al., Microbiol. Mol. Biol. Rev., 69(1):124-154, 2005.
Desvaux et al., Appl. Environ. Microbiol., 66:2461-2470, 2000.
Eriksen et al., Biotechnol. Lett., 30:103-109, 2008.
Fan et al., J. Power Sources, 171:348-354, 2007.
Greene et al., Int. J. Syst. Bact., 47:505-509, 1997.
Hammerschlag, Environ. Sci. Technol., 40:1744-1750, 2006.
Holmes et al., Microb. Ecol., 48:178-190, 2004.
Jong et al., Environ. Sci. Technol., 40:6449-6454, 2006.
Kashefi and Lovley, Science, 301(5635):934, 2003.
Kim et al., Enz. Microb. Technol., 30:245-252, 2002.
Liu et al., Environ. Sci. Technol., 38:2281-5, 2004.
Liu et al., Environ. Sci. Technol., 38:4040-6, 2004.
Liu et al., Environ. Sci. Technol., 39:5488-93, 2005.
Logan et al., Environ. Sci. Technol., 36:2530-2535, 2002.
Logan et al., Environ. Sci. Technol., 40: 5172-5180, 2006.
Logan et al., Environ. Sci. Technol., 40:5181-5192, 2006.
Logan, Water Sci. Technol., 52:31-37, 2005.
Lovley, Nat. Rev. Microbiol., 4:497-506, 2006.
Lovley, Nat. Rev. Microbiol., 4:497-508, 2006.
Lynd et al., Curr. Opin. Biotechnol., 16:577-583, 2005.
Lynd et al., Microbiol. Mol. Biol. Rev., 66:506-577, 2002.
Madigan et al., Curr. Opin. Microbiol., 2:265-269, 1999.
Madigan et al., In: Brock Biology of Microorganisms, 11^{th} Ed., Prentice Hall, 2005.
Mathis et al. Appl. Microbiol. Biotechnol., unpublished
Melis and Happe, Plant Physiol., 127:740-748, 2001.
Milliken and May, Appl. Microbiol. Biotechnol., 73:1180-1189, 2007.
Min et al., Environ. Sci. Technol., 38:5809-14, 2004.
Min et al., Water Res., 39:4961-4968, 2005.
Miroshnichenko et al., Inter. J. Syst. Evol. Microbiol., 53:1637-1641, 2003.
Morris et al., Electrochem. Comm., 9:1730-1734, 2007.
Niessen et al., Lett. Appl. Microbiol., 41(3):286-290, 2005.
Park et al., Appl. Environ. Microbiol., 66:1292-1297, 2000.
Potter, Durham Univ. Soc., 3:230-234, 1912.
Potter, Proc. Roy. Soc., B84:245-49, 1910.
Prince and Kheshgi, Crit. Rev. Microbiol., 31:19-31, 2005.
Qiao et al., Appl. Cat. B: Environ, 170:79-84, 2007.
Rabaey et al., Env. Sci. Technol., 39:3401-3408, 2005.
Rabaey et al., Appl. Environ. Microbiol., 70:5372-5382, 2004.
Rabaey et al., Environ. Sci. Technol., 39:3401-3408, 2005.
Rabaey et al., ISME J, 1:9-18, 2007.
Reimers et al., Environ. Sci. Technol., 35:192-195, 2001.
Ren et al., Environ. Sci. Technol., 41:4781-4786, 2007.
Rismani-Yazdi et al., Biotechnol. Bioeng., in press, 2007.
Rosenbaum et al., Appl. Cat. B. Environ., 74:261-269, 2007.
Schell et al., Appl. Biochem. Biotechnol., 105-108:69-85, 2003.
Shin et al., Appl. Microbiol. Biotechnol., 58:476-481, 2002.
Sokolova et al., Int. J. Syst. Evol. Microbiol., 55:2069-2073, 2005.
Takagi et al., Proceedings of the Bioconversion Symposium IIT, Delhi, 551-571, 1977.
Takai et al., Inter. J. Syst. Evol. Microbiol., 53:839-846, 2003.
Tender et al., Nat. Biotechnol., 20:821-5, 2002.
Tender et al., Nat. Biotechnol., 20:821-825, 2002.
ter Heijne et al., Environ. Sci. Technol., 40:5200-5205, 2006.
Wang et al., Appl. Environ. Microbiol., 73:5261-5267, 2007.
Wang et al., Philos. Trans. R. Soc. Lond. Ser., B300:323-333, 1983.
Wilkinson, Autonomous Robots, 9:99-111, 2000.
Yan et al., Appl. Environ. Microbiol., 73:5347-5353, 2007.
Yu et al., J. Power Sources, 171:275-281, 2007.
Zararzina et al., Extremophiles, 11:1-7, 2007.
Zhao et al., Electrochem. Comm., 7:1405-1410, 2005.

What is claimed is:

1. An isolated organic acid-consuming thermophilic electricigenic bacterium.

2. The isolated bacterium of claim 1, wherein the bacterium is Thermincola ferriacetica.

3. A process for generating electricity comprising supplying a microbial catalyst and a fuel source to a microbial fuel cell under thermophilic conditions, wherein the microbial catalyst consumes the fuel source and generates electricity.

4. The process of claim 3, wherein the fuel source is acetate or cellulose.

5. The process of claim 3, wherein the microbial catalyst is resistant to autoclaving.

6. The process of claim 4, wherein the microbial catalyst comprises Thermincola.

7. The process of claim 4, wherein the microbial catalyst comprises Deferribacteres.

8. The process of claim 3, wherein the microbial catalyst comprises more than one bacteria.

9. The process of claim 8, wherein the microbial catalyst comprises Thermincola and Deferribacteres.

10. A process of generating ethanol and electricity comprising:
(a) supplying a microbial catalyst and a fuel source to a fermentation vessel in operable relation with a microbial fuel cell, wherein the microbial catalyst has a cellulolytic activity, an ethanologenic activity, and an electricigenic activity; and
(b) wherein the fuel source is consumed and ethanol and electricity are generated.

11. The process of claim 10, wherein the fuel source is cellulose, hemicellulose or chitin.

12. The process of claim 11, wherein the cellulose is corn stover, peach waste, wood chips, forest litter, or switchgrass.

13. The process of claim 10, wherein the fermentation vessel is maintained under thermophilic conditions.

14. The process of claim 13, wherein the microbial catalyst comprises Clostridium thermocellum, Thermoanaerobacterium thermosaccharolyticum or Thermincola.

15. The process of claim 13, wherein the microbial catalyst comprises two or more bacteria selected from the group Clostridium thermocellum, Thermoanaerobacterium thermosaccharolyticum and Thermincola.

16. A process of generating ethanol and electricity comprising:
(a) supplying a microbial catalyst having ethanologenic activity and a fuel source to a first fermentation vessel, wherein a spent fuel source is generated; and
(b) supplying the spent fuel source and a second microbial catalyst having a cellulolytic activity and an electricigenic activity to a second fermentation vessel, wherein the second fermentation vessel is in operable relation with a microbial fuel cell, wherein said spent fuel source is consumed,
wherein ethanol and electricity are generated.

17. The process of claim 16, wherein the first and second fermentation vessels are maintained under thermophilic conditions.

18. The process of claim 17, wherein the microbial catalyst having an ethanologenic activity is Zymomonas mobilis and the fuel source contains both sugar and cellulose.

19. A process of generating hydrogen comprising supplying a microbial catalyst and a fuel source to a BEAMR system, wherein the microbial catalyst consumes the fuel source and generates hydrogen, wherein the BEAMR system is under thermophilic conditions.

20. The process of claim 19, wherein the fuel source is acetate or cellulose.

21. The process of claim 19, wherein the microbial catalyst is resistant to autoclaving.

22. The process of claim 20, wherein the microbial catalyst comprises Thermincola.

23. The process of claim 20, wherein the microbial catalyst comprises Deferribacteres.

24. The process of claim 19, wherein the microbial catalyst comprises more than one bacteria.

25. The process of claim 24, wherein the microbial catalyst comprises Thermincola and Deferribacteres.

26. A process of generating ethanol and hydrogen comprising supplying a microbial catalyst and a fuel source to a BEAMR system, wherein the microbial catalyst has a cellulolytic activity, an ethanologenic activity, and an electricigenic activity, wherein the fuel source is consumed and ethanol and hydrogen are generated.

27. The process of claim 26, wherein the BEAMR system is maintained under thermophilic conditions.

28. The process of claim 27, wherein the fuel source is cellulose, hemicellulose or chitin.

29. The process of claim 28, wherein the cellulose is corn stover, peach waste, wood chips, or forest litter.

30. The process of claim 26, wherein the microbial catalyst comprises Clostridium thermocellum, Thermoanaerobacterium thermosaccharolyticum or Thermincola.

31. The process of claim 26, wherein the microbial catalyst comprises two or more bacterium selected from the group Clostridium thermocellum, Thermoanaerobacterium thermosaccharolyticum and Thermincola.

32. An apparatus for generating ethanol and electricity comprising a fermentation vessel in operable relation with a microbial fuel cell.

33. The apparatus of claim 32, wherein the microbial fuel cell comprises an anode and at least two cathodes.

34. The apparatus of claim 33, wherein the microbial fuel cell comprises at least two anodes and a cathode.

35. The apparatus of claim 32, further comprising a inflow line and a return line, wherein the inflow line and the return line communicate between the fermentation vessel and the microbial fuel cell.

36. The apparatus of claim 32, further comprising a microbial catalyst.

37. An apparatus for generating ethanol and hydrogen comprising a chamber, a fuel source, a microbial catalyst, and a power source in connective relation to an anode and a cathode, wherein the anode and the cathode are located within the chamber.

38. The apparatus of claim 37, wherein the power source is a second microbial fuel cell.

39. The apparatus of claim 37, wherein the system further comprises a membrane.

40. The apparatus of claim 37, further comprising a microbial catalyst.


SEQUENCE LISTING (2 sequences)

SEQ ID NO: 1 - DNA, 20 bp, Artificial
  Synthetic primer
agagtttgatcmtggctcag

SEQ ID NO: 2 - DNA, 19 bp, Artificial
  Synthetic primer
ggytaccttgttacgactt